(19) United States
(12) Patent Application Publication
(10) Pub. No.: US 2010/0204459 A1
(43) Pub. Date: Aug. 12, 2010

(54) SYSTEMS AND METHODS FOR PRODUCING MULTI-COMPONENT COLLOIDAL STRUCTURES

(75) Inventors: Thomas G. Mason, Los Angeles, CA (US); James N. Wilking, Los Angeles, CA (US)

Correspondence Address: VENABLE LLP, P.O. BOX 34385, WASHINGTON, DC, 20043-9998, US

(73) Assignee: The Regents of the University of California, Oakland, CA (US)

(21) Appl. No.: 12/670,913

(22) PCT Filed: Aug. 27, 2008
(86) PCT No.: PCT/US2008/010120

§ 371 Date: Jan. 27, 2010

Related U.S. Application Data
(60) Provisional application No. 60/935,700, filed on Aug. 27, 2007.

(51) Int. Cl. B01J 19/00; C07H 21/04; C07K 1/00; B05C 11/00; B05D 7/00; C25D 5/00

(52) U.S. Cl. 530/408; 422/129; 422/119; 422/105; 204/600; 536/23.1; 530/391.1; 118/713; 427/212; 205/80; 204/450

(57) ABSTRACT

A system for producing multi-component colloidal structures has a supply system; an assembly system that is in fluid connection with the supply system to receive a supply of colloidal structural components from the supply system; and an output system in fluid connection with the assembly system. The assembly system has an assembly chamber adapted to contain colloidal structural components during assembly of a multi-component colloidal structure and is structured and arranged to control positions and orientations of first and second c structural components in the assembly chamber to bring the first and second colloidal structural components together in predetermined relative positions and orientations for assembly into at least a portion of the multi-component colloidal structure.

### CROSS-REFERENCE TO RELATED APPLICATION

This application claims priority to U.S. Provisional Application No. 60/935,700 filed Aug. 27, 2007, the entire contents of which are hereby incorporated by reference.

This invention was made using U.S. Government support under Grant No. CHE0450022 awarded by the National Science Foundation. The U.S. Government has certain rights in this invention.

### BACKGROUND

1. Field of Invention

The present invention relates to systems and methods for assembling multi-component colloidal structures, and more particularly systems and methods for assembling multi-component colloidal structures using optical trapping and manipulation.

2. Discussion of Related Art

All references cited anywhere in this specification are incorporated herein by reference.

Although an approach of using depletion attractions in combination with surface roughness on microscopic and sub-microscopic structural components (e.g. colloidal particles) dispersed in a viscous liquid offers a potential advantage of creating many identical assemblies of these microscopic and sub-microscopic structural components in a highly parallel manner, in some cases, for example for particles or subassemblies that become significantly larger than a micron in size, the diffusion times can be very long for the added parts to encounter and bind to form the assemblies properly. Thus, it may be desirable in some cases, for example where assembly driven by diffusion is particularly slow, to directly manipulate the particles and physically rotate and position them so that they approach and bind in the desired manner quickly. Optical methods of exerting forces and torques have been previously used to manipulate a very few basic shapes. However, a suitable system for building complex colloidal assemblies out of a wide variety of particle shapes using methods that permit the precise bonding together of components in a wide range of positions and orientations has not been developed. Moreover, due to the competition between destabilizing radiation pressure and stabilizing gradient forces that depend sensitively on the geometry and shape of the particles, it is not obvious that existing optical methods can be employed to position, much less orient, a wide variety of complex shaped dielectric components that are subject to thermal fluctuations in a fluid. Indeed, optical methods for manipulating particles have not yet provided arbitrary control over relative positions and angles of complex shapes of individual particles or complex subassemblies of particles that would be necessary to precisely build very complex colloidal assemblies, including machines with moving parts.

A single-beam field-gradient optical trap (Ashkin A., Dziedzic J. M., Bjorkholm J. E., and Chu S., Opt. Lett. 11 (1986) 288), known as ‘laser tweezers’, has been used to trap simple structures and to provide a limited degree of manipulation. A variety of microscale dielectric objects, such as spheres (Id.), cubes, rods, disks, and crosses (Higurashi E., Sawada R., and Ito T., Appl. Phys. Lett. 73 (1998) 3034; Gauthier R. C., Ashman M., and Grover C. P., Appl. Opt. 38 (1999) 4861; Cheng Z., Chaikin P. M., and Mason T. G., Phys. Rev. Lett. 89 (2002) 108303; and Galajda P. and Ormos P., Opt. Express 11 (2003) 446; respectively) have been trapped and manipulated to a limited degree using laser tweezers, however systems and methods for trapping and manipulating a variety of objects and bringing more than one of such objects together for assembly have not been conventionally available. Complex topological surfaces can be reduced to a Reeb graph that contains basic components that permit an object having a complex shape to be skeletonized and characterized in a systematic fashion (K. Cole-McLaughlin, H. Edelsbrunner, J. Harer, V. Natarajan, and V. Pascucci. “Loops in Reeb Graphs of 2-Manifolds” In Proceedings of the 19th ACM Symposium on Computational Geometry (SoCG), 2003, pages 344-350). The advent of methods for producing dielectric colloidal particles that have complex shapes (e.g. as revealed by non-trivial Reeb graphs characterizing the shapes) therefore presents a challenge as to whether such particles can be stably trapped, much less manipulated and positioned in a highly precise manner that would be necessary to build a precision assembly.

In the absence of optical absorption, dielectric particles near the focal point of a laser beam experience forces and torques arising from photon momentum transfer (Laser tweezers in cell biology, edited by Sheetz M. P. (Academic Press, Orlando) 1998 Vol. 55). Light that is backscattered from the particles creates an effective radiation pressure that pushes the particles in the average direction of propagation, k, of the laser beam. However, strongly focused laser light creates a very high electric field gradient in all directions around the focal point, resulting in a force that tends to pull a higher dielectric constant material into the region of highest field strength, even along k. Likewise, torques can arise from simple photon momentum transfer (Galajda P. and Ormos P., Opt. Express 11 (2003) 446; Galajda P. and Ormos P., Appl. Phys. Lett. 78 (2001) 249) and also from angular momentum transfer for optically anisotropic materials (Higurashi E., Sawada R., and Ito T., Appl. Phys. Lett. 73 (1998) 3034; Cheng Z., Chaikin P. M., and Mason T. G., Phys. Rev. Lett. 89 (2002) 108303; Friese M. E. J., Nieminen T. A., Heckenberg N. R., and Rubinsztein-Dunlop H., Nature 394 (1998) 348). If the forces and torques generated by radiation pressure overcome the gradient forces and torques that tend to stabilize the particle in the trap, then the particle will not trap and will be ejected in the k-direction away from the focal point. However, if at least one configuration can be found in which the forces and torques arising from radiation pressure and field gradients form a potential well that is significantly deeper than thermal energy k_{B}T, then the particle can be trapped stably in three dimensions.

The sizes and shapes of microscale particles play an important role in determining their stability and their potential positions and orientations in an optical trap. Simple rod-shaped particles are known to trap with their symmetry axes aligned along k (Gauthier R. C., Ashman M., and Grover C. P., Appl. Opt. 38 (1999) 4861), whereas thin disk-shaped particles and platelets are known to trap “on edge” with their symmetry axes aligned perpendicular to k (Cheng Z., Chaikin P. M., and Mason T. G., Phys. Rev. Lett. 89 (2002) 108303). In either case, the position and orientation of the particle maximizes the highest dielectric constant material in the region of the strongest electric field. Although much recent activity has been centered on patterning light in more complex ways for optical micromanipulation (Grier D. G., Nature 424 (2003) 810; Sinclair G., Jordan P., Courtial J., and Padgett M., Opt. Express 12 (2004) 5475; Dholakia K. and Reece P., Nano Today 1 (2006) 18; Huisken J., Swoger J., and Stelzer E. H. K., Opt. Express 15 (2007) 4921; Mohanty S. K., Dasgupta R., and Gupta P. K., Appl. Phys. B 81 (2005) 1063; Tanaka Y., Hirano K., Nagata H., and Ishikawa M., Electron. Lett. 43 (2007) 412), systems and methods for trapping and manipulating objects and bringing more than one of such objects together for assembly are not conventionally available. There is thus a need for improved systems and methods for assembling multi-component colloidal structures.

### SUMMARY

A system for producing multi-component colloidal structures according to an embodiment of the current invention has a supply system; an assembly system that is in fluid connection with the supply system to receive a supply of colloidal structural components from the supply system; and an output system in fluid connection with the assembly system. The assembly system has an assembly chamber adapted to contain colloidal structural components during assembly of a multi-component colloidal structure and is structured and arranged to control positions and orientations of first and second colloidal structural components in the assembly chamber to bring the first and second colloidal structural components together in predetermined relative positions and orientations for assembly into at least a portion of the multi-component colloidal structure.

A method of producing multi-component colloidal structures according to an embodiment of the current invention includes controlling a position and an orientation of a first colloidal structural component; controlling a position and an orientation of a second colloidal structural component; and bringing the first and second colloidal structural components together into predetermined relative positions and orientations to be assembled into at least a portion of a multi-component colloidal structure.

### BRIEF DESCRIPTION OF THE DRAWINGS

Additional features of this invention are provided in the following detailed description of various embodiments of the invention with reference to the drawings. Furthermore, the above-discussed and other attendant advantages of the present invention will become better understood by reference to the detailed description when taken in conjunction with the accompanying drawings, in which:

FIG. 1A shows a schematic illustration of a system for producing multi-component colloidal structures according to an embodiment of the current invention;

FIG. 1B shows views of a microfluidic container structure according to an embodiment of the current invention;

FIG. 2 shows an example of a microfluidic container structure that provides side port injection into the interaction region according to an embodiment of the current invention;

FIG. 3 shows an example of a complex container structure that facilitates the assembly of building-block particles using optical manipulation according to an embodiment of the current invention;

FIGS. 4A-4C illustrate a system and method for producing multi-component colloidal structures according to an embodiment of the current invention;

FIG. 5A-5D illustrate a system and method for producing multi-component colloidal structures according to another embodiment of the current invention;

FIG. 6 is a schematic illustration of a dual focused beam optical trap apparatus as an optical system according to another embodiment of the current invention;

FIGS. 7A-7D illustrate a system and method for producing multi-component colloidal structures according to another embodiment of the current invention;

FIGS. 8A-8C illustrate a system and method for producing multi-component colloidal structures according to another embodiment of the current invention;

FIG. 9 illustrates a system and method for producing multi-component colloidal structures according to another embodiment of the current invention;

FIG. 10 illustrates a system and method for producing multi-component colloidal structures according to another embodiment of the current invention;

FIG. 11 illustrates a system and method for producing multi-component colloidal structures according to another embodiment of the current invention;

FIG. 12 illustrates a system and method for producing multi-component colloidal structures according to another embodiment of the current invention; and

FIG. 13 provides schematic illustrations of some examples of methods for creating attractive interactions (also called ‘bonds’) between building blocks that can be stronger than thermal energy so an assembly of building blocks will remain together after being brought into close approach according to some embodiments of the current invention.

### DETAILED DESCRIPTION OF VARIOUS EMBODIMENTS

There are many ways of manipulating microscopic particles that are dispersed in a fluid (usually a viscous liquid). These manipulation methods include magnetic, fluidic, optical, electro-optical, electrophoretic, dielectrophoretic, and osmotic methods, to name a few. Although magnetic manipulation can be used to manipulate certain isolated particles or to form chains of particles, it is limited to a subset of particles that contain iron oxide nanoparticles or some other magnetically active material. Also, it is difficult to localize magnetic fields to orient and position nearby particles (i.e. interacting and not isolated) in an arbitrary manner. Likewise, electrophoretic and dielectrophoretic methods can cause the migration, concentration, and chaining of charged and dielectric particles, but only in limited ways, and individual manipulation of differently shaped particles to a wide range of configurations in close proximity is also quite difficult. In fluidic methods, arranging the fluid flow fields to cause two or more particles to combine in a prescribed way is possible, yet hydrodynamic interactions between the flow and the particles in close proximity are complicated and not always predictable. So, while making assemblies with any of these methods is possible, certain methods can offer advantages and disadvantages relative to the others.

For the purposes of building assemblies in some particular applications, which requires the manipulation of two or more complex shapes in close proximity, optical methods can offer the best combination of local control over the positions and orientations of nearby particles. As we show in some of the examples below, it is possible to trap many different dielectric shapes in a wide variety of positions and orientations. The trapping state obtained depends on the initial entry of the particle into the trap. Further manipulation is possible using polarization of the light to orient or even spin the particles. By using different color laser light (i.e. light having different wavelengths λ) to trap and manipulate different particles, interference of different manipulating beams can be avoided. This provides a means to manipulate particles in close proximity using different color beams of light without the added complication of controlling the interference of the optical fields as the particles approach. In some cases of optical manipulation, such as with holographic laser tweezers, the interference of laser light at only one wavelength is actually used to advantage in order to create a three dimensional light field that can be altered in many desirable ways.

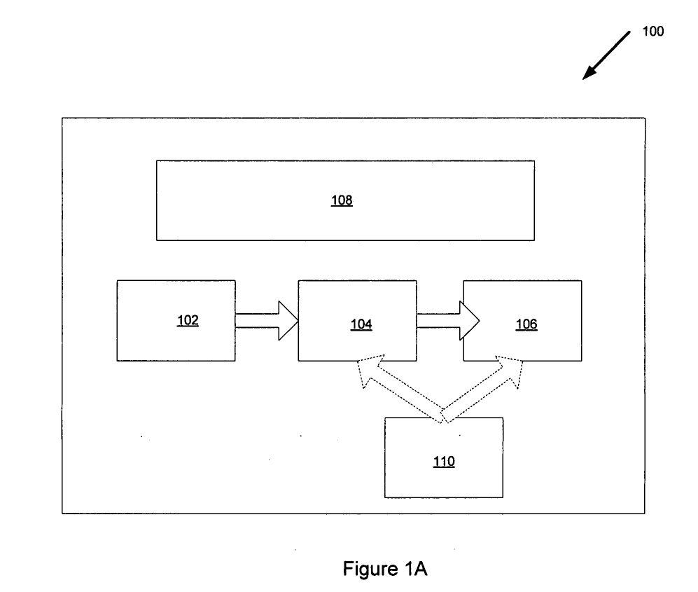

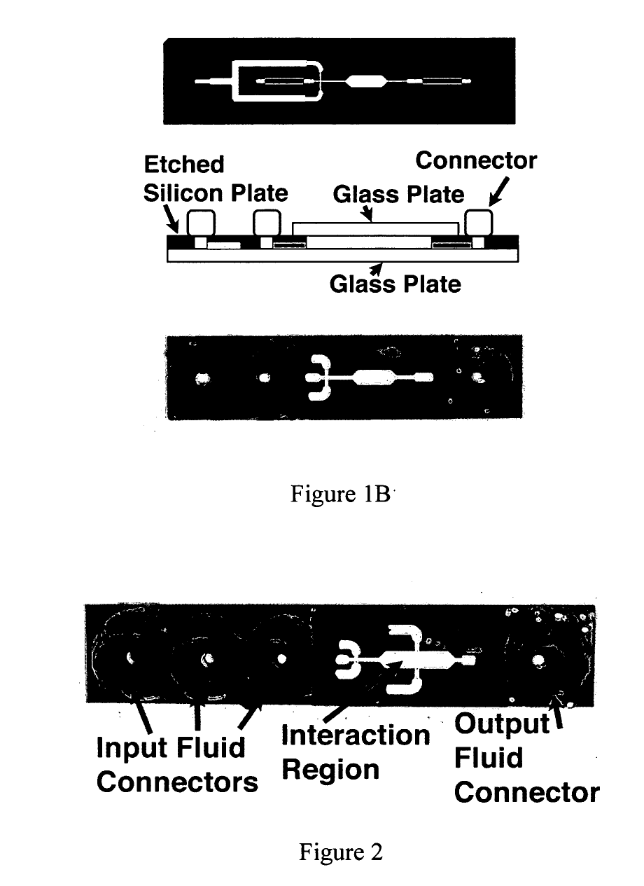

FIG. 1A is a schematic illustration of a system for producing multi-component colloidal structures according to an embodiment of the current invention 100. The system for producing multi-component colloidal structures 100 has a supply system 102, an assembly system 104 that is in fluid connection with the supply system to receive a supply of colloidal structural components from the supply system 102, and an output system 106 in fluid connection with the assembly system 104. The assembly system 104 has an assembly chamber adapted to contain colloidal structural components during assembly of a multi-component colloidal structure and is structured and arranged to control positions and orientations of first and second colloidal structural components in the assembly chamber to bring the first and second colloidal structural components together in predetermined relative positions and orientations for assembly into at least a portion of the multi-component colloidal structure. The assembly system can contain one or more sources of photon intensity that can be structured in space and time for manipulating first and second colloidal structural components into desired positions and orientations The system for producing multi-component colloidal structures 100 can also include an imaging system 108 constructed and arranged to observe, recognize, and record the positions and orientations of colloidal structural components in at least one of the supply system 102, the assembly system 104 and the output system 106 while the system for producing multi-component colloidal structures 100 is in operation. The system for producing multi-component colloidal structures 100 can also include a bonding material injection system 110 in fluid connection with the assembly system 104. The system for producing colloidal structures 100 and the associated subsystems can be controlled by a master computer system that coordinates the activities of the subsystems to provide an assembly-line step-wise automated production of multi-component colloidal structures.

Optical manipulation can be achieved using many specific methods involving the shaping of the light and adjusting its phase to trap, move, spin, and orient particles to a limited degree. Some examples below show how to manipulate a wide variety of differently shaped particles using the simplest form of optical manipulation: the single beam gradient trap resulting from a single focused laser beam, according to an embodiment of the current invention.

Although the principles presented herein are demonstrated with particles that typically have dimensions greater than or equal to the wavelength of the light used for optical manipulation, it is likely that many of the processes will still be effective in creating assemblies even when the particles become comparable to or even somewhat smaller than the wavelength of light. There may be some changes to the precise details of how to control the positions and orientations of the particles compared to the larger particles, but it is highly likely that the general principles will hold. However, as the particles become very small compared to the wavelength of light in the manipulating field, the degree of control and localization of the particles may not be as great. In such cases one can reduce the wavelength of the light in order to improve the manipulation. This could mean changing the wavelength of the source of electromagnetic radiation from visible light to ultraviolet (UV) light, to deep ultraviolet (DUV) light, or even to use soft x-rays. (The terms “light” and “optical” are not intended to be limited to particular wavelengths of electromagnetic radiation. In some embodiments, it can be electromagnetic radiation in the visible region of the spectrum. However, it can also refer to light in non-visible regions of the electromagnetic spectrum such as infrared, UV, DUV or soft x-ray regions, for example.) Using strictly monochromatic light is not a necessity for the manipulation according to some embodiments of the current invention. However, there can be many advantages to using the monochromatic coherent light of laser sources in some embodiments of the current invention. Moreover, according to some embodiments of the current invention, it can be advantageous for the suspending fluid to neither strongly absorb nor scatter the electromagnetic radiation.

Herein, we use the word assembly to refer to a structure comprising two or more building blocks that are bound together. We also refer to the building blocks as colloidal structural components and the assemblies as multi-component colloidal structures. Herein, the use of the term “colloidal” is intended to mean length scales that can roughly extend from several nanometers up to and even well beyond 10 microns, although most common definitions limit this upper length scale to about a few microns. Assemblies can include structures in which building blocks are bound through shear-rigid and/or slippery bonds. Thus, assemblies can have both static and moving parts. It is conceivable that microscopic and nanoscopic assemblies, such as structures, devices, and machines, including examples such as engines, motors, propellers, crawlers, wheels, containers, valves, and pumps, for example, can be made through a process of controlled assembly of tiny building blocks. Useful processes for arranging building blocks in a fluid into useful assemblies at the microscopic scale are not conventionally known for any arbitrarily shaped objects that have dimensions of about ten microns or less. Merely scaling down conventional systems and methods of macroscopic construction (e.g. assembly-line robotics) to the colloidal scale has not been demonstrated, primarily due to problems with any and all of the following: making colloidal complex components, manipulating these colloidal structures (i.e. components) into proximity with a desired relative position and orientation, and binding these components together in the desired positions and orientations with a desired type of bonding. Thus, when considering the prior art, it is not obvious how a wide range of desired complex assemblies could be manufactured. Herein, in one of the embodiments of the current invention, we overcome this limitation through a manufacturing systems that consists of a combination of sub-systems that are properly coordinated to produce the desired complex assemblies.

General Principles of Building Custom Colloidal Assemblies

Colloidal Building Blocks in a Fluid

To build an assembly one must be provided with at least two building blocks, generally of colloidal dimension (e.g. from about 30 nm to about 100 microns). These building blocks are generally particles comprised of a condensed phase material. The material of each building block could be solid, viscoelastic, liquid crystalline, or a liquid. In general the material of which the building block is comprised must be able to remain for a time in the fluid long enough for the assembly to be created. It must not dissolve so rapidly that the process of creating the assembly cannot be carried out. The material can be brittle, flexible, or viscoelastic. The material can potentially swell and shrink. A building block can be comprised of a single material or a plurality of materials; such material(s) include but are not limited to: dielectric solids, polymer materials, composite materials, nanoparticulate materials, magnetic materials, optically absorbing materials, optically non-absorbing materials, optically chiral materials, fluorescing materials (e.g. dye molecules and quantum dots), imaging enhancing materials (e.g. for magnetic resonance imaging (MRI), positron emission tomography (PET) imaging, optical assays, x-ray tomographic imaging), inorganic materials (e.g. silicon oxide, silicon nitride), organic materials (e.g. homopolymers, diblock copolymers, tri- and multi-block polymers, crosslinked polymers, derivatized polymers, waxes, resins, rubbers, photoresists, epoxy photoresists), metal-inorganic materials, metallic materials, conducting materials, inorganic-organic materials (e.g. metal organic frameworks), biological materials (e.g. DNAs, RNAs, oligomeric DNAs, oligomeric RNAs, proteins, oligopeptides, block copolypeptides, polysaccharides), catalytic materials, liquid materials, viscoelastic materials, photoreactive materials, chemically reactive materials, heat-sensitive materials, heat reactive materials, dissolvable solid materials, bioreactive materials (e.g. enzymes), biomolecular assemblies, cellular biological structures, sub-cellular biological structures, biological organelles, biological cells, and biological tissues.

Generally, the building blocks can be dispersed in a fluid. This fluid can be a simple viscous liquid, but it could also be a gas, a supercritical fluid, a plasma, a suspension of nanoparticles, a suspension of nanodroplets, a multi-phase dispersion, an ionic liquid, or even a viscoelastic material or complex fluid, such as a polymeric liquid. The fluid can be a mixture of several different miscible fluids. Immiscible fluids could also be used in special circumstances where the assembly occurs at an interface between the immiscible fluids.

These building blocks (also referred to as colloidal structural components) can be custom-shaped particles that are produced lithographically through top-down means, for example, or they can be any form of colloidal object that is produced through top-down processes, bottom-up processes, or a combination thereof. By bottom-up processes, we refer to, for instance, self-assembly of atomic, molecular, cluster, or nanoparticulate materials. Bottom-up processes also include phase separation and spinodal decomposition or other methods of introducing controlled heterogeneity of materials that can be used to create a building block. Building blocks can be separate, non-contiguous objects. Building blocks do not usually dissolve in the fluid while an assembly is being built, but it can be possible and even desirable for a particular building block to be dissolved during the process of building a complex assembly. In such a case the building block is a “temporary building block”, whereas building blocks that can remain for very long periods of time in the fluid without being degraded or otherwise altered are called “permanent building blocks”. In most cases, the building blocks are permanent.

Building blocks may be referred to as parts, components, pieces, colloidal structural components or particles of which an assembly is comprised. Even liquid building blocks, such as droplets, may be called colloidal structural components hereafter.

Examples of building blocks include colloidal particles that have been designed and fabricated by lithographic means (e.g. photolithography, dip-pen lithography, imprint lithography, and other lithographic fabrication methods), microspheres (e.g. obtained from controlled emulsion polymerization), microparticles, wax microdisks, emulsion droplets, nanoparticles, liquid crystalline droplets, nanoparticles, quantum dots, and microscale and nanoscale crystallites and clusters.

Building blocks may be comprised of more than one phase of materials. A building block may be comprised of a solid particle that has liquid components either on the surface and/or inside. These liquid components can potentially be reactive, as in the case of an epoxy or glue.

The surfaces of these building blocks (i.e. particles) can be modified in ways to create specific surface roughness, coatings, and binding sites and chemistries for controlling surface-mediated interactions between the particles. Portions of the surfaces of building blocks can be modified using different materials to provide surface-specific or site-specific attractive or repulsive interactions. Materials that can be used to modify the surfaces of building blocks can include: a surfactant, an anionic surfactant, a cationic surfactant, a zwitterionic surfactant, a non-ionic surfactant, a polymeric surfactant, a lipopolymer, a lipid, a lipid bilayer, a lamellar vesicle, a multi-lamellar vesicle, a polymer, a derviatized polymer, a homopolymer, a copolymer, a block copolymer, a random copolymer, a polymer brush, a polymer coil, a polymer tether, a star polymer, a dendrimer, a polyacid, a polybase, a polyelectrolyte, a peptide, a copolypeptide, a multi-block peptide, a semiflexible polymer, a flexible polymer, a polyethylene glycol, a polysaccharide, a polyhydroxystearic acid, a polyvinylalcohol, a polysiloxane, a charge group, a sulfate group, a sulfonate group, a carboxylate group, an amine group, an acidic group, a basic group, a biomolecule, a biopolymer, a derivatized biopolymer, an antibody, an antigen, a peptide, a polypeptide, a copolypeptide, an amino acid, a protein, a membrane protein, a transcription protein, a structural protein, a viral protein, a snare protein, an actin, a tubulin, an enzyme, a vitamin, a biological cell wall, an albumin, a collagen, a cellulose, a cholesterol, a biomolecular motor, a kinesin, a saccharide, a polysaccharide, a liposaccharide, a biotin, an avidin, a streptavidin, a nucleic acid, a ribonucleic acid, a deoxyribonucleic acid, a derivatized deoxyribonucleic acid, an oligomeric nucleic acid, an oligomeric single-stranded deoxyribonucleic acid, an oligomeric double-stranded deoxyribonucleic acid, a biomolecular assembly, a biomotor, an acidic material, a basic material, a metallic material, an inorganic material, an organic material, a polar material, a non-polar material, a particulate material, a microparticle, a nanoparticle, a droplet, a microdroplet, a nanodroplet, a chemically reactive material, a thermally reactive material, a photoreactive material, a photoabsorbing material, a catalytic material, an isotopic material, a radioactive material, an imaging enhancing material, a thiolated molecule, an alkane, a silane, a siloxane.

Container Structure

It is conceivable that a container is not necessary to create assemblies. For instance, if one places a bead of liquid containing building blocks directly on the lens of a microscope objective, it may be possible to manipulate particles and create an assembly optically. However, this would usually be inconvenient, since removing the assemblies would be problematic and the objective would have to be cleaned. In most cases, the completed assembly must be delivered somewhere beyond the point of construction to be useful, so a container structure is typically provided.

The fluid and building blocks (particles) in the fluid are usually contained in a structure that can support the fluid mechanically and inhibit fluid evaporation. This can also be referred to as an assembly chamber without limitation on the size and shape of such a containment region. Although it is not necessary, it is usually desirable for the structure to be transparent to the form of electromagnetic radiation that is being used to manipulate the particles. Such container structures can include: thin glass plates, slides, cover slips, wafers, solid films, flexible films, liquid films, membranes, dialysis membranes, capillary tubes (with any cross section—circular, square, rectangular), capillary plates, microwell plates, hard microfluidic devices, and soft microfluidic devices. Examples of materials of which the container structure can be constructed in whole or in part include: glass, quartz, sapphire, silicon, metals, organic materials, inorganic materials, plastic materials, polymeric materials (e.g. polydimethylsiloxane—PDMS), reactive materials, epoxies, thermally curable adhesives, and UV-curable adhesives.

It can be desirable to modify the surfaces of the container chemically or physically to prevent the particles and assemblies from sticking to the container's surfaces. For instance, in the case of depletion attractions used to hold particles together, it may be necessary to roughen the surfaces of the container structure in contact with the fluid to prevent particles from sticking to the walls of the container structure. In other cases, it may be desirable for a building block or assembly to stick to a part of the container surface, so coating a portion of the container's surface with a material that promotes the adhesion of the building blocks or assemblies may be desirable.

Container structures can also be comprised of electrodes, opto-electrical transduction layers, magnetic materials, or any other materials that can potentially be used to assist in modifying pure optical manipulation of the particles.

Microfluidic devices can offer many features that make them a particularly useful container structure. Microfluidic devices can contain reservoirs, storage cavities, input channels, exit channels, cross-channels, constrictions, expansions, pumps, valves, capillary tubes, connectors, and other structures. They can be equipped with external tubing, pumps, valves, and storage locations to facilitate the manipulation of the fluid surrounding a desired particle, assembly, device, or machine. This fluid manipulation of the building blocks and the assemblies can occur prior to, concurrent with, and subsequent to the assembly of two or more building blocks.

The container structure of a microfluidic device can offer many advantages over other containers for a number of important reasons. These can include:

(1) Building blocks of a known type can be stored in separate reservoirs that have particular well-defined locations, so the location of a source of a particular particle type (e.g. a particle having a specific size, shape, and composition) is known.

(2) Building blocks of a known type can be brought into rough proximity with other building blocks of a known type through the use of flow. The number of a specific building block per unit time can even be metered and adjusted relative to the timing of the metered flow of other building blocks. This can overcome a potential need to precisely control the optical fields over very large distances in order to move particles from a reservoir region to an interaction region. The precise optical manipulation required for assembling building blocks is usually needed only over a relatively small interaction region.

(3) Specific interaction regions can be connected in sequence in the same container structure so that many stages of an assembly process can be carried out more easily in the same container. Thus, one can make a microfluidically-assisted “assembly line” in a single container structure.

(4) Many such microfluidically-assisted assembly lines can be fabricated on a single microfluidic device. This can enable parallel assembly so that the same manipulations can be done in multiple places simultaneously to increase the throughput.

There are some constraints on using microfluidic devices as container structures for some embodiments of this process. To facilitate flow of the building blocks and assemblies, the dimensions of the microfluidic channels and cavities can be designed to be larger than the largest lateral dimension of the building blocks (for entry channels) and of the final assembly (for the exit channels). Otherwise, the particles or assemblies could get stuck in the channels, creating an obstruction that precludes the proper operation of the device.

Many methods can be used to construct microfluidic container structures. Anodically bonding two flat glass plates onto opposite sides of a patterned and etched flat silicon wafer to create a layered sandwich structure can be a convenient method for creating a robust container structure that has storage regions, microchannels for transporting building blocks and assemblies, and interaction regions. This container structure can provide facile optical access to all of these regions from two sides. This method of creating hard microchannels can be especially appropriate when the fluid is a viscous liquid. Another method of constructing a microfluidic structure involves patterning PDMS on the surface of one transparent solid (e.g. a glass slide).

In most but not all cases, one can modify the surfaces of the container structure that particles may encounter in such a manner that the particles do not stick to the surfaces irreversibly. This can be achieved by treating the surfaces or coating the surfaces of the container structure with a non-stick material. Other surface treatments can involve depositing dielectric layers (e.g. for use as anti-reflection coatings), a layer of indium tin oxide (e.g. for use as a conductive layer), or even increasing the surface roughness through etching.

FIGS. 1B, 2, and 3 show some examples of container structures. FIG. 1B shows an example of a microfluidic container structure according to an embodiment of the current invention. The top view is a schematic illustration of a pattern that is created as a mask for performing lithography on a silicon wafer (dark) that typically has a thickness between 50 microns and 1 mm and is double polished. The white regions indicate where the wafer is etched completely through. The grey regions indicate locations where the wafer is etched only partially through on one surface, usually about halfway through. Two different fluid inputs (left) are injected into a cross channel that, along with the wide slit channel (middle), comprise the interaction region. The middle view in FIG. 1B is a side view schematic illustration of a microfluidic channel along the centerline of the device. The silicon wafer is anodically bonded between two glass plates. The glass and silicon plates may be thin, so the absolute and relative thicknesses of the plates are not to scale. The grey regions are etched through partially to provide a fluid conduit between the connector and the interaction region. The bottom of FIG. 1 is a top view photograph of an actual microfluidic container structure suitable for making assemblies through optical manipulation according to an embodiment of the current invention. The overall length of the device is 40 mm and the silicon wafer and corresponding channels are 250 microns in height.

FIG. 2 is an example of a microfluidic container structure that provides side port injection into the interaction region (photograph—top view) according to an embodiment of the current invention. A silicon wafer has been lithographically patterned, etched, and anodically bonded between two glass plates. Connectors have been attached to the silicon to facilitate transport of fluid materials that can contain building blocks, sub-assemblies, and assemblies into the microfluidic device. Three different input fluid materials can be directed to the central interaction region by flow through microfluidic channels. At least one of these fluid materials contains building blocks, and all of the fluids may contain particulates or chemicals that can be used to bond the particles together once they are brought into close approach by optical means in the interaction region, or “assembly chamber”. The interaction region provides good optical access from two different directions through the optically transparent top and bottom glass plates according to this embodiment of the current invention. Optical and fluidic manipulation of building blocks injected from the input channels can be used to cause close approach of the building blocks. A fluid material that promotes binding of closely proximate particles can be injected through the side channels. After bonding, the assembly made of building blocks can be moved to the output reservoir and out of the reservoir into microfluidic tubing attached to the connector. The overall length of the channel is about 45 mm. The height of the silicon wafer, corresponding to the thickness of the fluid region in the channel, is 250 microns.

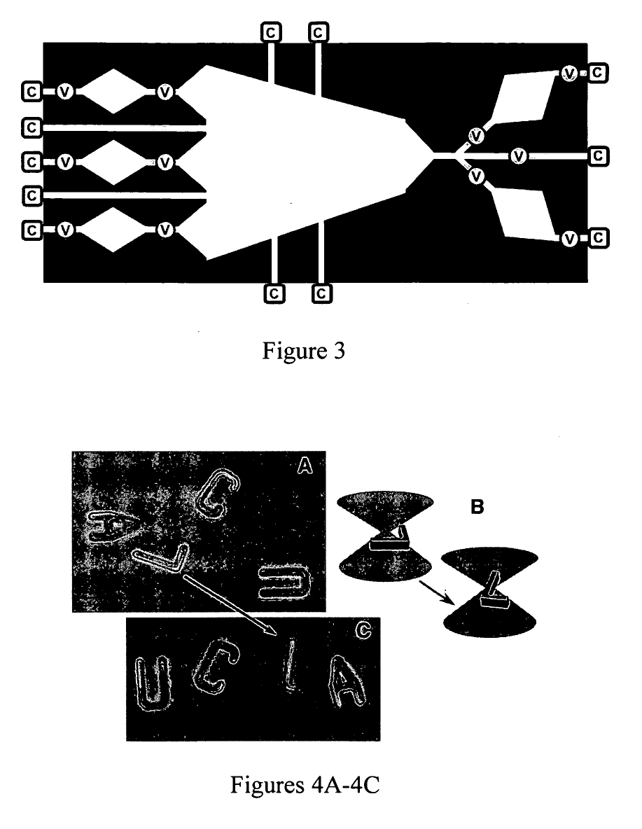

FIG. 3 is a schematic illustration of an example of a design for a complex container structure that facilitates the assembly of building-block particles using optical manipulation according to another embodiment of the current invention. This is a top view of a microfluidic device showing the layer of solid material that is sandwiched between optically clear plates. Microfluidic connectors (C) and valves (V) enable the fluids containing building blocks, assemblies, and/or other materials to be loaded into the container structure, stored in the container structure, manipulated in the container structure, reacted in the container structure, and expelled from the container structure. The valves and connectors are optional, but they can provide greater control over storing the building blocks and completed assemblies. Building blocks can be stored in the diamond shaped-reservoirs on the left side of the structure as shown. These building blocks can be controlled to enter the larger interaction region in the center. The precise optical manipulation to cause close approach of building blocks occurs in the interaction region. Input ports and side ports may be used to provide materials that permit bonding of the particles to occur. Output reservoirs (tilted diamonds on the right side) can be used to store the completed assemblies. Multiple copies of this container structure can be connected in series and in parallel to provide high-throughput sequential step-wise addition of building-block components (e.g. particles or sub-assemblies) to an assembly.

Locating Particular Building Blocks

Locating a particular desired building block is a necessary prerequisite for manipulating it near other building blocks and building an assembly. This can be achieved in several ways.

One method is to use real-time video microscopy and image analysis of shapes of many differently shaped particles diffusing in the fluid. By analyzing microscopy images of the particles (as two dimensional projections using standard microscopy and in three dimensional voxel images using confocal microscopy), it is possible to identify and locate a particular shape. Such video image analysis can be performed in real time and used in a feedback loop with any manipulation system to confirm the location of the particle.

Another method is to use a reservoir in a microfluidic device or connected to a microfluidic device that is filled with building blocks having a pre-determined shape in the fluid. The location of the reservoir or input channel thus uniquely determines the shape of the particle that can be introduced. These two methods can be combined so that particle recognition software, used in combination with microscopic video imaging, is employed simultaneously with microfluidic flows of several building blocks to locate those that will be manipulated and organized to form the assemblies. Reservoirs can have valves or doors that can release a certain particle of a particular type into an interaction compartment or into a microchannel that can be used to flow the particle into the interaction region. These valves or doors can be mechanically actuated constrictions that physically prevent the escape of certain particles from uniquely identifiable reservoirs. Valves and doors can also effectively be created using electric, magnetic, or electromagnetic fields that would block the escape of particles out of the reservoirs. It is usually desirable for the building-block particles in the reservoirs to be un-aggregated and well dispersed.

Electrophoretic bottles can be used to hold certain building blocks in specific locations in an open chamber without typical microfluidic channels; physical barriers are not necessary to keep certain particles in a pre-determined location so that identification is not needed to be certain that a certain desired shape will be properly selected.

Moving Building Blocks into an Interaction Region (Assembly Chamber)

After the building blocks have been located, they can be moved into a region in the container structure (or from the container structure into an interaction structure if it is separate from the container structure) where the actual positioning and manipulation of the building blocks into close proximity takes place.

Diffusion of building blocks from the reservoir region into the interaction region is generally slow, but it is a possible method of transport to get building blocks into an interaction region.

A much faster and usually more desirable way to move the building blocks into the interaction region is through microfluidic flows. Fluid flows can be used to convectively transport one or more building blocks from the reservoir storage regions in the container structure into the interaction region. Fluid flows can be created by various kinds of pumps, by applying a fluid pressure, by suction, by vacuum, by a microturbine, or by any other means of generating a flow. Any particles in the fluid are swept along by the viscous drag of the fluid into the desired region. An optical microscope equipped with a computer controlled video acquisition system can be used to monitor the location of the particles in the fluid and stop the flows when the particles reach the interaction region by commanding a computer-controlled pump connected to the computer to stop. This optical verification system is not necessary, but it may be useful and convenient.

Some advantages of using microfluidic flows for this step can include:

(1) It is simple to precisely coordinate the movement of many differently shaped particles into the interaction region once the microfluidic device has been designed and constructed.

(2) A wide range of particle materials can be transported in this manner by viscous drag forces and fluid flow.

(3) Particles can be transported over distances that are many times larger than their sizes.

(4) Viscous drag can be used to transport particles over a very wide range of sizes, including nanoscale particles.

Another means of moving particles into the interaction region is through electrophoresis. By applying an electric field to charged particles, it is possible to induce them to move.

Dielectrophoresis can be used to move uncharged (neutral) dielectric particles. The electric fields can be computer controlled and connected by feedback to the same computer that does video microscopy to monitor the positions and orientations of particles according to some embodiments of the current invention.

Magnetically responsive building blocks can be moved by magnetic fields and magnetic field gradients into the interaction region.

Optical manipulation of the building blocks can also be used to bring particles into the interaction region. Single-beam laser tweezers, multi-beam laser tweezers, holographic laser tweezers, interfering multi-spot beam patterns, opto-electric methods are some that could be used to trap and move the particles from the reservoir region to the interaction region. Each of these methods has different strengths and weaknesses. For instance, with single beam laser tweezers, one can manipulate the particles by moving the microscope stage upon which the container structure rests while keeping the laser beam position fixed. Alternatively, one can fix the position of the container structure and move the laser beam, or a combination of both moving the stage and the beam. Thus, according to some embodiments of the current invention, a computer-controlled motorized microscope stage, potentially including an embedded computer-controlled piezoelectrical microscope stage, which typically holds the container structure, can be incorporated into the assembly system.

Precise manipulation of the particles is not needed for the gross transport of particles from the reservoir regions into the interaction region. The main purpose is to bring the building blocks into a region of about 100 microns× about 100 microns× about 100 microns without letting them potentially touch and aggregate in an uncontrolled manner before the more precise optical manipulation can take over and direct their assembly.

Unless diffusion is used to cause the transport, the strength (e.g. forces and torques) of the method of manipulation to move the particles in a directed manner must be significantly stronger than those associated with thermal fluctuations.

Manipulating the Position and Orientation of at least one Building Block

Once the building blocks are in the interaction region, it can be necessary to precisely control the position and orientation of at least one building block in the process of creating an envisioned and desired pre-determined assembly structure, or at least a sub-structure of the total desired assembly structure. Thus, it can be desirable to define methods of manipulation of the position and angle of a single colloidal particle in a fluid.

In most cases, the center of mass of the particle can be used for the purposes of identifying the particle's position. The center of mass of an object is commonly defined in introductory physics textbooks.

In some trivial cases, such as manipulating a uniform dielectric sphere, the orientation of the particle is indistinguishable and usually unimportant. In general, the shape of the building block determines how many distinguishable angles are needed to define the orientation of an object. These angles are referred to as ‘Euler angles’, and it is commonly known that three Euler angles are necessary to describe the orientation of an object having an arbitrary shape. In navigation terminology, these angles are generally called tilt, yaw, and roll. More symmetric shapes may not require as many angles. A desirable form of manipulation of a single particle would provide control over three position coordinates (x, y, z) and three angle coordinates (θ, α, β), regardless of the particle's shape or internal material composition. These position coordinates and angle coordinates are sometimes referred to collectively as “coordinates” of a particle. Although many forms of manipulation of a variety of shapes have been demonstrated using optical tweezers and other methods, the problem of manipulation of colloidal objects remains open to advances scientifically, since no single manipulation method has been demonstrated to independently manipulate all positional coordinates and orientation angles for all shapes over the total possible valid range that these may take.

For custom-shaped particles, it is desirable to have a general method to manipulate them in a variety of positions and orientations that overcomes the constant buffeting of thermal fluctuations that is the origin of Brownian motion. This requires that the applied forces and torques used to hold and turn the particles must be at least as strong as the average Brownian forces and torques. In most cases, it is desirable for the applied forces and torques, which fix a particle's position and orientation, to be much stronger than the Brownian forces and torques.

The following list provides a guide of optical methods that can be used to manipulate single particles in enough coordinates for building an assembly according to some embodiments of the current invention:

Electromagnetic radiation pressure (photon backscattering) from a beam of light incident on a particle: although this cannot be used by itself to trap a particle, it can be used to propel a particle in a desired path towards another particle that has been identified. Generally, this path is along the average direction of the propagation of the photons.

Counter-propagating beams of light: radiation pressure can be used to trap and move a particle in a controlled manner. Counter propagating beams have their average propagation directions parallel, but with the opposite sign (i.e. direction).

Simple gradient optical trap formed by a single focused light beam (“laser tweezers”): a high numerical aperture lens is typically used to focus a beam of light, creating a region near the focus where the electromagnetic field radiation is stronger and the light intensity is brighter. Typically, the lens also has high magnification. The region of high intensity has a strong gradient in the electric field strength in all directions around the brighter focal spot. This gradient acts to move dielectric particles that have a higher refractive index toward the spot, and particles that have a lower refractive index tend to be moved away from the spot. If the light intensity is sufficiently high and if the gradient forces overcome the forces due to radiation pressure, then a particle can be stably trapped and moved in (x, y, z). This motion can be made by moving the position of the focal spot and also by moving the container structure relative to the focal spot. Means of focusing the light can include: lenses, mirrors, microlens, micromirrors, fiber optical graded refractive index (GRIN) lenses, Fresnel lenses, and diffractive lens optics (e.g. zone plates).

Many variations of a simple gradient optical trap for holding particles exist. Some permit a limited degree of manipulation of the angles and positions of the particles. These variations can include modification of the following properties of the light beam or beams:

Spatial structure of the electromagnetic radiation (light): Laser modes of all orders and indices (Gaussian; Laguerre-Gaussian; Hermite-Gaussian). For instance, it can be advantageous to increase the relative proportion of photons that are focused at high angles (e.g. taking full advantage of the high numerical aperture of the objective lens) by directing most of the laser intensity to form a donut-like mode (i.e. lower intensity along the optical axis and higher intensity in a ring surrounding the optical axis).

Beam spatial structure: circular and elliptical beams, Elliptical profile created by a cylindrical lens, “Line tweezers” and “Star tweezers” made by combining several line tweezers that have different orientations, Two or more beams of light, Counter-propagating beams, Multiple beam injection into a focusing element, Microlens arrays, Micromirror arrays, Acousto-optical modulators (spatial light modulators), Multi-beam interference, Interfering coherent light, Holographic laser tweezers, Electronically-addressable acousto-optical modulators, Electronically-addressable micromirror arrays, Permanent phase plates and masks, Focused beams of spatially patterned light, Shadow masks create a patterned beam that is focused, This patterned beam interacts and traps a dielectric particle, Rapid motion of a region of intense electromagnetic radiation, Scanning or rastering a focal spot rapidly, Piezoelectric elements (computer controlled) move spot, Polarization of the light;

Spin angular momentum: Linear polarization, Random polarization, Elliptical polarization, Circular polarization;

Orbital angular momentum: Helical beams, Polarization can be used to align and spin trapped particles;

Spectral structure of the light: Monochromatic (e.g. laser emits photons at one wavelength), Bi-chromatic (two discrete wavelengths), Multi-chromatic (multiple discrete wavelengths), Polychromatic (continuum of wavelengths), Single line emission, Single frequency emission;

Coherence of the light: Coherent, Partially coherent, Incoherent;

Temporal dependence of the light: Continuous illumination, Periodic pulsed illumination, Intermittent illumination, Computer-controlled illumination

The source of electromagnetic radiation can be one of a laser, a laser diode, a light emitting diode, an optical parametric oscillator, an optical resonator, a lamp, a bulb, a filament, a plasma, or a chemical reaction. These devices all come in many variations.

In an example of a single beam gradient trap, a Gaussian beam of linearly polarized transverse electromagnetic TEM₀₀ light from a monochromatic laser is introduced into a focusing element (e.g. a high 1.4 numerical aperture (NA) microscope objective lens), the laser beam is expanded to fill the back aperture of the objective lens. The degree of beam expansion can affect the spatial profile, and a highly expanded Gaussian beam that is larger than the objective's aperture can be truncated, leading to an alteration of the spatial structure of the focused light. Beam expansion and truncation can play an important role in the strength of the optical trap. In some cases, index-matching immersion liquids (e.g. oil, water) are optionally needed to reduce undesirable scattering and reflections that can occur between the objective lens and the optical windows of the container structure.

Light wavelengths that can be used to trap colloidal particles range from about 100 nm to about 3 microns. In principle, even shorter wavelengths could be used, but care must be taken so that the focusing optics and fluid material can permit light propagation at such short wavelengths. These limitations have generally been overcome in devices such as lithography steppers.

Certain kinds of Laguerre-Gaussian beams can be used to trap particles that have a refractive index that is smaller than the fluid. The principle behind this approach is that the particle can be trapped inside the donut-shaped light, which creates a repulsive barrier around the particle in all three dimensions, provided the radius of the donut is larger than the particle.

For optical manipulation of a building block, there must be a way of introducing the light into the interaction region. This can be achieved by making at least a portion of the container structure transparent. It can also be achieved by inserting fiber optics into the container structure. Fiber optics can transport light in very tiny spaces with low loss and they can be equipped with a focusing lens at their ends, which can be used to trap, move, and orient particles.

Optical forces and torques generated by these methods can be used in combination with any of the following methods of generating forces and torques to further control the position and orientation of the building block: fluidic (e.g. generated through fluid flows, including flows in microfluidic devices), viscous, magnetic, electrophoretic, dielectrophoretic, induction, osmotic, buoyant (i.e. gravitational), and thermal.

The confinement of particles by solid surfaces of the container structure can also be used to aid in the manipulation and approach of the particles. For instance, if the particles do not irreversibly bind to the surface of the container when they approach it, then the walls of the container can be used to limit the range of motion of the particles. Particles manipulated by optical forces near the walls of the container can achieve a different orientation than in the bulk fluid. This can be used to advantage in making assemblies.

A way of trapping and manipulating a particle that has a complex shape and possesses a higher refractive index relative to that of the fluid into a desired position and orientation is to rapidly move the location of a brightly illuminated spot (e.g. near the minimum beam waist of a highly focused laser beam coming from a high numerical aperture microscope objective lens) to create a time-averaged trapping potential energy landscape that strongly favors having the particle move and rotate so that its higher dielectric constant material occupies the regions of brightest illumination. This method relies on the known behavior that higher refractive index materials are attracted to regions of greater light intensity. Rather than using a static spot that is in one location or rather than just translating the spot slowly, a rapid motion of the spot can be achieved using piezoelectric elements to translate, deflect, and otherwise move the beam rapidly before it enters the focusing objective lens. When used in combination with actuators (e.g. piezoelectric elements) that move the microscope's objective, the stage upon which the container structure rests, or even change the degree of divergence, convergence, or expansion of the laser beam entering the objective, full three-dimensional control over the laser spot can be achieved over a range of distances from several nanometers to hundreds of microns. This range is appropriate for trapping and manipulating complex building blocks that have sizes from about ten nanometers to tens of microns. With fast actuators such as piezoelectric elements, it is possible to move the bright spot in a trajectory that effectively traces out the shape of a complex object in only a few milliseconds, so the process of moving the beam over the entire shape of the object is typically repeated many times per second. By systematically altering the ‘tracing’ trajectory swept out by a focused laser spot, the complex shape can be moved and rotated into a desired position and orientation. Tracing the laser spot in the focal plane perpendicular to the optical axis can be accomplished by piezoelectric mirrors, yet tracing out of the focal plane could also be desired. To simultaneously accomplish this tracing, altering the location of the laser spot along the optical axis of the microscope objective can be accomplished by slightly under or overfocusing the light introduced into the microscope objective (e.g. through a beam expander). All of the devices used to manipulate the focal spot to ‘trace out’ the particle shape in its desired position and orientation can be controlled and coordinated by a computer.

Coordinating shuttering (i.e. blocking) of the light beam in synchronization with the elements that move the beam can provide even greater range of options for creating a complex light field. This can be achieved by having electronic shuttering elements and power drive electronics for the beam actuation (e.g. piezoelectric elements) connected to an electronic device, such as a computer, that provides control signals that can have precise time coordination. If the motion of the laser spot is fast enough, shuttering can provide a method that can trap more than one particle using a single beam. This can be achieved by opening the shutter so the beam is transmitted, rapidly moving the beam using the piezoelectric elements to move the laser spot in the form of the first desired shape at the desired position and with the desired orientation, rapidly closing the shutter so that the beam is blocked, rapidly moving the beam to a different desired location for a second particle but the laser spot does exist during this operation because the beam is blocked from entering the objective lens, rapidly opening the shutter so that the beam is transmitted, rapidly moving the beam using the piezoelectric elements to move the laser spot in the form of the second desired shape at the second desired position and with the second desired orientation. This method can further be extended to trap and manipulate a plurality of particles that have complex shapes. As the same optical source is shared among many different spatial locations, it is usually desirable to ensure that the power of the source is sufficient to provide trapping forces and torques that are significantly larger than those associated with equilibrium thermal fluctuations.

For instance, a static focused beam can be used to trap a particle in the shape of the letter H (which has a higher refractive index than the fluid outside it) as shown in FIG. 1 of “Multiple trapped states and angular Kramers hopping of complex dielectric shapes in a simple optical trap” by J. N. Wilking and T. G. Mason, EuroPhysics Letters, 81 (2008) 58005, the entire contents of which are incorporated herein by reference. The H particle traps on its side with the crossbar pointing in the average propagation wavevector k (i.e. direction) of the laser beam. This restricted degree of control alone could be used to significant advantage to control the particle for building an assembly. However, the range of angular orientations of the particle is limited. The H particle could be manipulated in an angle in the plane perpendicular to the propagation direction of the laser through light polarization or even by making the shape of the beam elliptical rather than circular. Although these refinements offer more possibilities for manipulation, they do not provide exact control over all three position coordinates and all three angle coordinates. They only provide control over three position coordinates and one angular coordinate. The other two angular coordinates would be fixed so that the H particle's crossbar points along k. However, if the very bright focused laser spot is rapidly moved to trace out the shape and size of the H particle to be trapped in a desired position and orientation, then the effective potential energy of the H particle in the complex optical potential that is shaped like the H will be minimized when the H particle becomes trapped in the position and orientation of the time-averaged brightest part of the laser illumination. Thus, by effectively drawing out the geometry of the particle shape to be trapped in the desired position and orientation in three dimensions, it is possible to obtain precise control over all three position coordinates and all three angle coordinates of very complexly shaped particles. Although tracing out the particle shape in a continuous sweep is one viable method, alternative related methods also exist, such as tracing out only a portion of the shape that is adequate to guarantee control. Alternatively, manipulating a limited set of at least three laser spots for a single particle, rather than tracing out the particle shape, could be used to provide adequate control over the position and orientation of the particle.

For dielectric particles that have a refractive index that is smaller than the fluid outside, the method of rapidly moving the bright spot could potentially be used to create a trap for the particle. In this case, the beam would not be moved in a form of the desired particle shape, but instead, it would be moved in a form to create a ‘cage’ outside the desired particle shape. Particle materials that have a lower refractive index than the fluid are repelled from regions of high light intensity, so the optical cage would effectively repel the particle from moving to locations outside the cage.

Typically, piezoelectric elements can provide beam motions at frequencies of up to about one-hundred kiloHertz, usually limited by the resonant frequency of the element. A more typical frequency would be from about one kiloHertz to ten kiloHertz. This method of rapidly moving a bright spot can still trap the particle in the desired manner even if the spot does not trace out the entire shape of the particle, but only enough of a subset of the shape of the particle so that the particle becomes trapped in the desired position and orientation according to some embodiments of the current invention. Using a microlens array illuminated by a laser beam having controlled collimation and direction, many particles could be trapped in many bright spots in precisely defined positions and orientations according to some embodiments of the current invention.

Holographic laser tweezers (also known as holographic optical trapping) can be used to create complex regions of localized bright illumination of coherent laser light that could also resemble the shapes or portions of shapes of complex particles according to some embodiments of the current invention. In this method, the interference of the laser light is controlled, typically using a spatially controlled acousto-optical modulator (AOM), otherwise known as a spatial light modulator (SLM). These devices typically operate by altering the local phase of the light that strike the device to pattern the amplitude and phase of the light at a point of reconstruction, usually after focusing the light (e.g. using a microscope objective lens) that has been modified by these devices. These devices generally have two-dimensional arrays of small discrete pixels that can independently adjust the phase of a coherent light beam locally. By altering the phase of the light in very localized regions in a controlled manner, it is possible to cause the light to interfere in a controlled way to create desired spatial patterns of higher and lower intensity. If this interference-based holographic method is used to create regions of bright illumination in the form of particle building blocks or portions thereof, then a building block (having higher refractive index than the fluid outside it) can be manipulated in all three position coordinates and all three angular coordinates. This is highly desirable for the purposes of manipulating a colloidal object as a part of a process to form a colloidal assembly. Using a computer or other electronic device to control the spatial phase adjustments of the light by the AOM or SLM, it is possible to reposition and re-orient the building block. Updating of the pattern on an AOM or SLM can be typically accomplished by sending a video signal (e.g. VGA or XGA) to the device, and this video signal can be generated in real time by a computer. As with the method of rapidly moving the laser spot, the method using holographic laser tweezers can offer a route to control and manipulate particles that have a very large range of complex shapes and sizes according to some embodiments of the current invention. As with the method of rapidly moving the laser spot, the method of using holographic laser tweezers can be used to trap a plurality of particles by forming two or more regions of bright light that have the form of the desired building blocks in different positions and orientations according to some embodiments of the current invention. Building blocks that have a lower refractive index than the fluid outside can be trapped by causing the holographic laser tweezers to form regions of bright intensity outside the building block that effectively cage it. The cage can be dynamically manipulated by adjusting the signals to the AOM and SLM to provide position and orientation control. It can be desirable for a control computer to coordinate signals to the SLM with signals to other computer-controlled devices in the other systems (e.g. systems that can produce microfluidic flows and systems that can induce binding).

Micromirror arrays can also be used to deflect and focus light, and these may be able to operate at higher light intensities than AOM's, yet achieve very similar spatial patterning of the light in a desired manner according to some embodiments of the current invention. Micromirror arrays are used to create high definition images for large television applications, and, through appropriate magnification, they could also be used to trap and manipulate particles. One method that can be used according to some embodiments of the current invention is to use a computer to create a video signal for a micromirror array in a method that is different than holographic laser tweezers. When the array is illuminated with an expanded laser beam, the reflected light can be collected and focused by a microscope objective into the interaction region of the container structure. This reflected light is comprised of many wavelets that have different directions, as introduced by slightly different angular positioning of the tiny micromirror elements. This creates a microscopic version of the commanded spatially patterned beam of light. By using the computer to command the micromirrors to create a region of bright intensity that resembles a particular shape of a particle in a desired position and orientation in three-dimensions, it is possible to trap and precisely manipulate the position and orientation of the particle according to some embodiments of the current invention.

With the methods of rapidly moving a bright spot, the method of holographic laser tweezers, and the method of micromirror arrays, it is possible to trap almost any dielectric shape, even the letter-shaped particles and particles with holes that we could not trap using a simple static focused beam in “Multiple trapped states and angular Kramers hopping of complex dielectric shapes in a simple optical trap” by J. N. Wilking and T. G. Mason, EPL, 81 (2008) 58005. The particles that did not trap at the particular laser power reported of 17 mW, such as K, V, X, and Y, as well as square frame particles could be trapped using the methods of rapidly moving a bright spot and the method of holographic laser tweezers, can all be trapped using these more sophisticated optical methods. The principle behind this is that the gradient forces can overcome the radiation pressure because the bright regions of the light are better matched in size and shape to at least an adequate portion of the particle.

In many cases, it is useful to know where particles are in the interaction region so that the methods to bring them in close approach are more efficient. For instance, the initial locations of the particles in the interaction region would provide very useful information that could be used to initially position the trapping and manipulation optical fields (or other methods of controlling the particles). Also, to prevent defects in the assembly structures, it is useful to verify that a particle has been trapped and has achieved a desired position and orientation that has been commanded without escaping the trap. This can be achieved using an optical microscopy apparatus that is equipped with a digital camera, digital video camera, or other similar sensing device. Often the same objective lens that is used to focus the laser light can be used to identify and track the positions and orientations of particles. Image analysis software can be used to quantitatively determine the position coordinates and angle coordinates. Most forms of optical microscopy are limited to two-dimensional projections or sections, but confocal microscopy can provide three-dimensional information. This imaging system can be connected to the control system for commanding the devices that structure the optical fields in order to trap and manipulate particles.

In some cases, it is useful to identify a particle using an optical microscopy apparatus equipped with an electronic camera and then use this information to command the construction of a particular trapping potential created by a particular optical method. This can be achieved by connecting the video microscopy system to a computer that can communicate with the control system for commanding the manipulation of the optical intensities, fluid flows, and other manipulation aspects.

An example of positioning structures using a single focused beam gradient optical trap, in combination with the constraints of a flat solid surface and viscous drag, is shown in FIG. 4. In particular, FIG. 4 shows an example of using a focused laser beam in combination with fluid viscosity and the presence of a solid surface to manipulate and write a desired sequence of letter-shaped building blocks to spell ‘UCLA’ according to an embodiment of the current invention. The helium-neon laser has a Gaussian spatial profile and a power of about 17 mW and is expanded and focused by an objective lens (Nikon CFI60 100×1.4NA). FIG. 4A shows building blocks (SU-8 epoxy resin particles) that do not initially have the desired structure. The building blocks are constrained by a flat solid surface, yet a lubricating layer of water remains between the particles and the surface. FIG. 4B is a schematic illustration of a single focused beam used to manipulate the letter-shaped building blocks. By trapping and translating the bright spot which holds the particle relative to the stationary fluid, viscous drag can be used to orient the particle so that its long dimension comes into alignment with the direction of translation. Generally, it is necessary to move the particle at least half its length for good alignment to be obtained, and more rapid motion causes better alignment, so long as the particle remains trapped. Moving the bright spot too rapidly can cause the viscous drag forces to become so large that the particle cannot keep up with the motion of the bright spot and is lost out of the trap. FIG. 4C shows that by blocking the laser beam, moving the microscope's mechanical stage, unblocking the laser beam, trapping another letter, moving it into a new position, and repeating, the entire sequence is created. Thermal agitation of the dielectric letter shapes gradually leads to some loss of alignment; this could be greatly reduced by rigidly bonding the particles to a film or another larger building block.

Causing Building Blocks to Closely Approach

Although direct manipulation of two or more building blocks can be a desirable method for causing two building blocks to approach in some embodiments, it is not a strict requirement in all embodiments of the current invention. In fact, it is possible to manipulate only one particle in a very limited way and yet cause it to move in a prescribed manner close to another particle. This can be sufficient to reproducibly create a desired relative position and orientation of two particles as a step in making an assembly. The particles may have similar or different shapes.

Herein, the terms ‘close approach’ and ‘close proximity’ mean that the surfaces of the two particles are separated by a distance that is small enough to enable bonding to occur between the particles for materials and/or processes that can cause bonding to occur.

Herein, the term ‘bringing into close proximity’ can have many meanings. It can mean at least one of bringing two particles side-by-side, face-to-face, side-to-face, inserting at least part of one particle into an opening in another particle, causing one particle to touch another particle physically, causing a specific surface or portion of a surface of one particle to be brought within a certain small distance from a specific surface or portion of a surface of another particle.

It is often convenient, but not a strict requirement, to carry out the close approach in a part of the container structure called the interaction region. Usually, this region provides optical access to the particles. This may involve making the container structure modular in design, and it may require a layer of solid transparent material to be present so that appropriate optical components (e.g. compound lenses for focusing light) can access at least a portion of the container structure for the optical manipulation methods to work properly. Many types of lenses have short working distances, so such layers of transparent material may need to be thin, and immersion oil or other index matching liquids may need to be present between the optical elements and the container structure.

One of the key aspects of this step in making an assembly of building blocks is using some method of microscopic or nanoscopic manipulation to overcome two important features present in colloidal dispersions: thermal fluctuations and viscous drag forces. To overcome thermal fluctuations, the effective potential energy well associated with the particle being in the desired position and orientation created by the electromagnetic fields must be significantly deeper than the thermal energy, k_{B}T, where k_{B} is Boltzmann's constant and T is the temperature. Since the potential well usually gets deeper and steeper as the intensity of the light is increased, this can usually be overcome by choosing an appropriately large light intensity. To overcome viscous drag, it is important for the optical restoring forces that tend to keep a particle in the brighter region of the light beam to be larger than the viscous drag forces from the fluid that resist any motion of that particle according to some embodiments of the current invention. If a bright region of intensity (e.g. focused spot) is moved in order to reposition a particle held in it, the viscous drag forces, which are generally at least proportional to the velocity, exerted by the fluid on the moving particle can become very large and can potentially exceed the trapping forces. In this case, the particle will lag behind the bright spot and can even escape the trap. Increasing the intensity of the light can increase the trapping forces resulting from photon momentum transfer, and can improve the rate at which the particle can be transported through the fluid while being held by the optical forces. Although viscous drag forces ultimately limit the rate at which a particle can be held and moved through the viscous fluid, for appropriately bright light in a weakly viscous fluid, the rate of motion can be quite large, exceeding the range of millimeters per second.

When two particles closely approach at a certain relative velocity, hydrodynamic interactions can occur that can affect the rate of approach. For the two particles to nearly touch, fluid must be transported away from the region between the particles through flow. This draining fluid flow between the particles could potentially prevent the particles from approaching closely enough to make a bond, since such flows involve the viscous resistance of the fluid. If the particles are not moved too rapidly together, it is possible for this fluid to move away from the region between the particles enough for the particles to approach closely. Therefore effective repulsive interactions between the particles that are created and mediated by viscous drag forces are not an inherent limitation, although they may place limits on the rates at which two particles can be brought into close proximity.

Once two or more particles closely approach, it may be necessary to reduce the light intensity once they are near each other. This can be accomplished by many means, including using a shutter to block the light completely or using other methods (e.g. gradient neutral density filter) to at least partially block the light from interacting with the building blocks. In one realization, a shutter can be used to block the light that has been used to optically trap particles from entering the interaction region, at least temporarily. In certain cases below, light from an optical trap holding one particle can exert undesirable radiation pressure on the particle that is being approached. This can occur even if the light beams being used to trap nearby particles have different wavelengths. Although the particles may still form an assembly even if they move out of the focal plane of the objective lens, it is usually desirable to keep the assembly in the focal plane for ease of observation.

Method 1: Radiation Pressure (Single Light Beam)

Radiation pressure can be used to drive one particle so that it closely approaches another particle. This method may be especially effective when used in combination with the walls of the container structure so that it has much lower mobility.

Method 2: Radiation Pressure (More Than One Light Beam)

Radiation pressure from two or more light beams can be used to push at least one particle into close proximity with another particle.

Method 3: Radiation Pressure (One or More Light Beams) with Fluid Flow

A combination of fluid flow and radiation pressure can be used to drive one particle into close approach with another particle. Converging flows in a microfluidic device can be a particularly good method of causing two or more particles to closely approach. This method may also be combined with the restriction of motion of particles by the walls of the container structure.

Method 4: Focused Light Beam—Two or More Particles in the Same Beam

Two or more particles that are in the same highly focused beam can move into the bright focal region where they will be concentrated. As a result, the two particles can closely approach, usually near the bright focal spot. In specific cases, it may be possible for certain shapes to create the same assembly reproducibly using this method.

As the degree of control over the relative positions and orientations of the building-block particles increases, so does the range of possibilities of the assemblies that can be made. Optical methods can offer the advantage that beams of light at two distinctly different wavelengths do not interfere, so it is possible to independently create many different local regions of bright illumination using light having different wavelengths. Once it is possible to trap and manipulate a single building-block particle in all three position coordinates and all three angle coordinates, then this property of light makes it reasonably straightforward to extend these methods to trap and manipulate a plurality of particles simultaneously and in nearby regions by simply employing light illumination at different wavelengths. This can be achieved, for instance, by making use of different laser lines from a single laser, or by using several different optical sources, such as lasers or optical parametric oscillators, to create light that has a plurality of wavelengths. Using more than one wavelength may be convenient, but it is not a strict requirement for manipulating more than one particle, since the intensity created by a single beam of monochromatic light can be structured in a manner to trap and manipulate two or more particles simultaneously in the interaction region. Some methods, such as holographic laser tweezers, rely upon the coherence of the light in order to pattern it in a desirable manner. Laser light sources can create either pulsed or continuous light output that can be used for creating optical forces and torques on particles.

The following methods offer a much greater degree of control over the relative positions and orientations of the particles according to some embodiments of the current invention.

Method 5: Gradient Optical Trap Comprising a Single Focused Light Beam

One focused Gaussian laser beam can be used to create a bright spot after passing through a microscope objective lens. For a single beam, a first particle can be trapped, repositioned, and reoriented (usually only to a limited degree with reorientation). Once this first particle is trapped, it can be brought into close proximity with a second particle, even if the second particle is not trapped. The first particle can be moved through the fluid near the second particle by moving the bright focused laser spot (e.g. by moving optical elements such as mirrors, lenses, and/or the laser source) or by moving the container structure relative to the brightly focused spot. The close approach of the two particles can also be accomplished without moving the first particle but just holding it; instead, by flowing the fluid in the interaction region to move the second particle into the first particle that is held in a fixed location by optical forces and torques. In this case, it is usually highly desirable to have some means of microscopically identifying the position and orientation of the particle that is not being trapped. This can also enable an automated system monitor and choose a particular pathway through which the particle to be tweezed enters the optical trap, which can affect its position and orientation in the trap (as described in detail in the manuscript (“Optically tweezing the colloidal alphabet”).

Method 6: Gradient Optical Traps Comprising a Plurality of Focused Light Beams

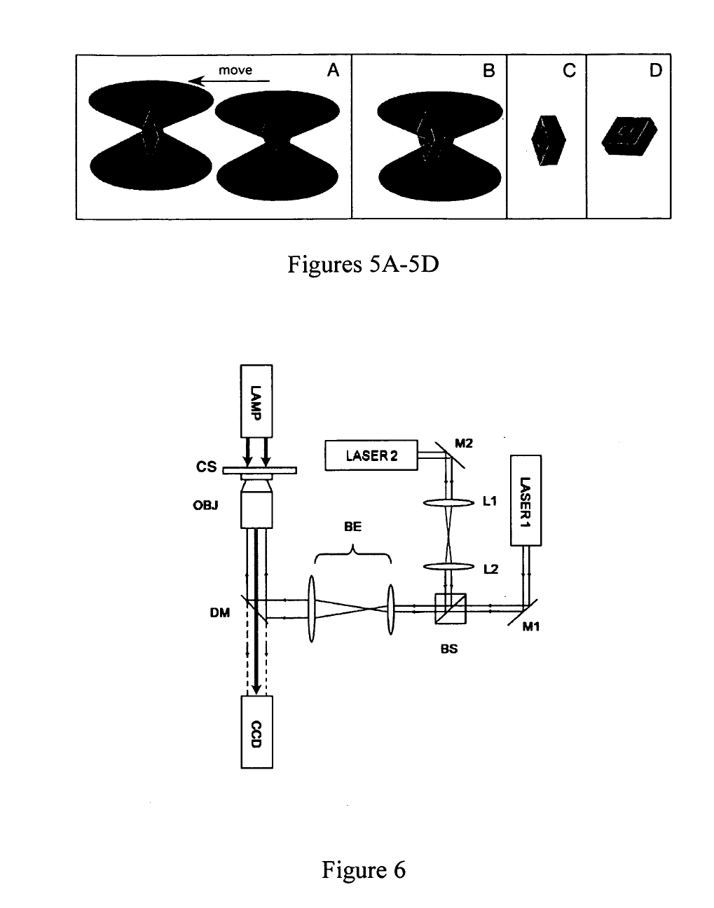

In this method, two or more beams of light, which may have the same or different wavelengths, are focused to create a plurality of optical traps. Two or more particles in the interaction region can be trapped and moved using procedures similar to those described in Method 5 above. These particles can be moved together in a particular sequence and order in order to achieve a desired assembly. This process potentially allows multiple copies of an assembly to be made in parallel using the same sequence. This method is illustrated in FIGS. 5 and 6.

FIG. 5 is a schematic illustration of an example of using optical manipulation to assemble a cup from two different microscale building blocks ('particles'), a square platelet particle and a square frame particle, using two focused laser beams. The two particles are in a viscous liquid that contains a depletion agent (e.g. nanoparticles or nanodroplets) in sufficient quantity to cause a strong depletion attraction. For clarity, these depletion agents are not shown. The particles have been transported to the interaction region where optical manipulation is facile. In FIG. 5A one focused laser beam traps a square platelet near its bright spot, and another traps a square frame that has a square hole in its center. Both beams are linearly polarized along the same direction to orient the building blocks so that their faces are parallel. The position of the laser spot holding the square frame platelet is moved toward the position of the laser spot holding the square platelet. This is done slowly enough as the particles near one another (i.e. come in close proximity) that fluid drag does not cause the square frame platelet to reorient significantly. In FIG. 5B, as the bright spot of the moving beam causes the square frame platelet to closely approach the square platelet, the static beam is blocked as the two platelets come together near the bright spot of the remaining focused beam. In FIG. 5C, as the face of the square frame bonds to a face of the square platelet due to the depletion attraction to create an assembly that is a cup, the second beam is blocked. The completed cup assembly diffuses in the suspension of depletion agent without coming apart (FIG. 5D).

Note that if the concentration of the depletion agent in the liquid is reduced significantly after step D, then the assembly could fall apart back into discrete building blocks, since the attraction would be greatly diminished. Other kinds of attractions than depletion attractions can be used to permanently bond the square frame to the square platelet.

FIG. 6 is a schematic illustration of a dual focused beam optical trap apparatus. Both lasers (LASER1, LASER2) emit beams that are directed by mirrors (M1, M2), combined using a beam splitter (BS), directed through a beam expander (BE, ˜6×), reflected by a dichroic mirror (DM) into the rear aperture of a microscope's objective lens (OBJ: e.g. 100× magnification, numerical aperture NA=1.4). The objective lens focuses the expanded laser beams, which are typically offset, into the container structure (CS) where the building blocks are in a fluid. The focused beams create two bright spots. A dichroic mirror reflects laser light but transmits light at a different wavelength from a light source (LAMP) so that the positions and orientations of building blocks can be seen by a video camera (CCD) that is connected to a computer that can analyze and record the images in real-time. Some residual laser light can leak through (dashed red lines) the DM; this can be filtered out before the CCD. M1 is controlled by a piezoelectric element connected to the computer that receives the video signals. The lenses (L1, L2) are used to keep M2 parfocal with the microscope objective. M2 could also be controlled piezoelectrically using the same computer to move both focused laser spots independently.

Method 7: Rapidly Moving the Bright Region (‘Spot’) of One or More Focused Light Beams

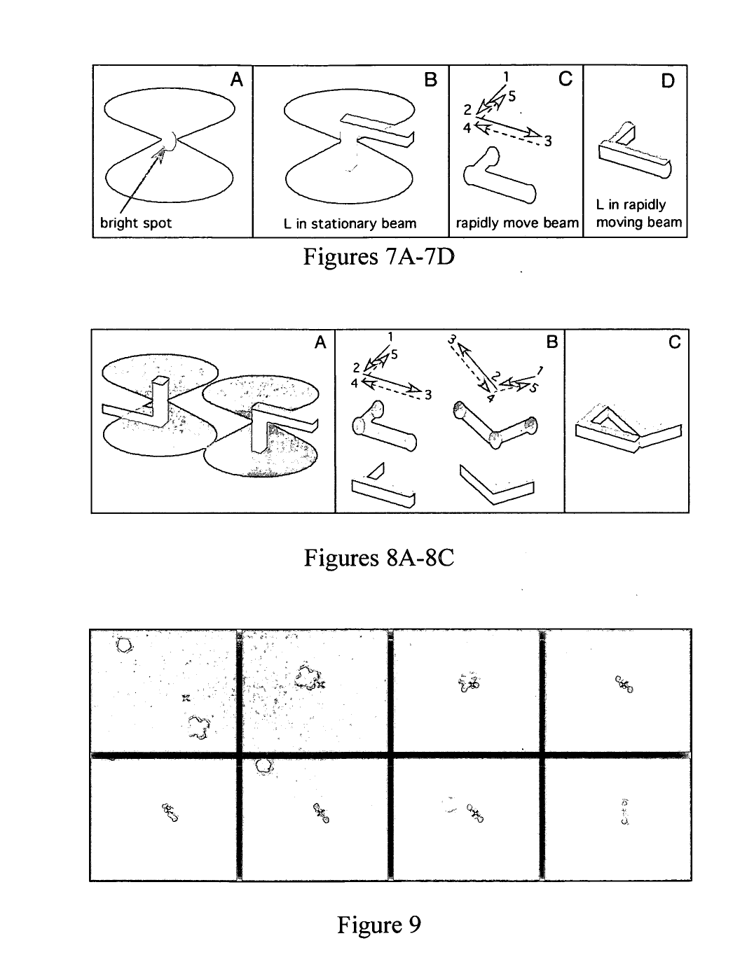

The bright spot of a single focused beam can be rapidly moved by means described above to trap, position, and orient particles having complex shapes. One particle can be moved close to another particle, which can also be held by a rapidly moving bright spot, even from the same source. In this case, the spot can be shared between two or more particles, with shuttering used as an option as the spot moves between the two different particles. The more a single spot is shared between many particles, the lower the dwell time of the spot with a given particle, so the optical forces and torques trapping and holding a particle become weaker. If a single spot is shared between many particles, it may be necessary to increase the laser power to compensate for this effect. By changing how the spot is moved (e.g. through computer control over piezoelectric elements) and how the beam is shuttered, it is possible to cause two or more particles to approach each other, where both particles have a desired position and orientation. Although sharing the same bright spot between many particles is one way of achieving this control, another method is to introduce a plurality of bright spots that are each independently moved and controlled. This can be achieved by providing several different laser sources (or by splitting a single laser beam into several weaker beams), and then moving the spots created by focusing each of these beams through deflection using separate piezoelectric elements. The same objective lens can be used to focus the beams into the interaction region. This method is illustrated in FIGS. 7 and 8.

As shown in FIG. 7, rapidly moving the bright spot of a focused laser beam can provide trapping and orientation control of complex building blocks. FIG. 7A shows that the bright spot of a focused laser beam can resemble an ellipsoid that has dimensions that are typically near the wavelength of light in directions parallel and perpendicular to the average direction of propagation k of the laser beam (here toward the top of the page). Usually the spot is elongated somewhat along k. As shown in FIG. 7B a building block in the shape of the letter L, when held in a stationary (non-moving) single focused beam optical trap, typically assumes this orientation. To rotate the building block into a different desired orientation, while still maintaining control over the position of its center of mass, the bright spot of the focused laser beam can be rapidly moved to trace out the shape in the desired position and orientation (FIG. 7C). Here, the beam is rapidly moved in the sequence shown (1 to 2 to 3 to 4 to 5, where positions 1 and 5 are the same and positions 2 and 4 are the same) to trace out the form of the L-shaped particle. Because the rapidly moved laser spot creates an effective time-averaged potential well in the shape of the L, the L rotates and comes in alignment with the moving laser spot to minimize its potential energy (FIG. 7D). For some building-block shapes, it may be necessary to initiate the motion of the bright spot gradually, rather than instantaneously, since an abrupt change in the trapping potential could cause the particle to leave the trap. This can be accomplished by moving the laser beam not just to trace out the final desired position and orientation of the building block, but to generate intermediate positions and orientations, too. Overall, this method of rapidly moving the laser spot in all three dimensions can be used to move and rotate building blocks that have a wide variety of shapes into a desired position and a desired orientation.

FIG. 8 shows an example of using two rapidly moving bright spots to rotate and align two building blocks with a high degree of control, facilitating the bonding of the building blocks into a desired assembly structure. FIG. 8A shows two building-block particles in the shape of the letter L that are trapped in static focused Gaussian TEM₀₀ beams and that take one of two stable orientations, with the short segment pointing up or down. Two different wavelengths (red light and green light) are shown here, but it is not necessary to use two different wavelengths for this process. Since the particles are not in the desired relative orientation and relative position, the bright spot of each focused laser beam is rapidly moved rapidly in order to position and align each of the particles as shown (FIG. 8B). Once the desired relative orientation is achieved, the patterns formed by the rapidly moved bright spots of one or both beams are translated in space to achieve the desired relative position. The particles held in these positions and orientations are bonded to form an assembly. In FIG. 8C, the assembly resulting from the optical manipulation of both building blocks has the desired structure. This method can also be used to insert arms of one building block into holes of another building block.

Method 8: Holographic Laser Tweezers

This method is similar to Method 7 above, except that holographic laser tweezers are used to position and manipulate one or more particles. After trapping the desired particles using brighter regions, the interference pattern is changed dynamically using an AOM, SLM, or other device to cause the particles to closely approach by moving the brighter regions together.

Method 9: Focusing a Beam of Light Patterned by a Mask

This method is also similar to Method 8 above, except that a shadow mask, phase mask, or other type of mask is used to pattern the light, rather than holographic laser tweezers. This pattern of light can be changed dynamically (e.g. by rotating sections of a mask into the laser beam to effectively create a movie of the desired position and orientation of at least one particle). The sequence of masks (or even moving one fixed mask) can be used to move at least one particle in close proximity with another. This method can be extended to create a plurality of trapping beams that can be used to make a plurality of the same assembly in parallel.

Method 10: Micromirror Arrays

This method is similar to method 8 above, except that micromirror arrays are used to create the desired regions of bright intensity in order to trap and re-orient at least one particle and bring it in close proximity with at least one other particle. A micromirror array controlled by a computer can cause desired dynamic changes in the light patterns that can facilitate moving at least one particle near another particle. More than one assembly can be created simultaneously in the same or in different interaction regions using this method.

Method 11: Opto-Electric Control

If the container structure is coated with an appropriate set of semiconducting, conducting, and dielectric materials, it can be possible to cause light to generate sufficiently strong electric fields near the surface coatings in order to cause the motion of particles. This method cannot offer the precise positioning and orientation of the particles that can be provided by some of the previous methods yet it is another possible method of causing the close approach of two particles.

Method 12: Rapidly Moving Many Bright Regions using Multi-lens Arrays

It is possible to apply method 5, method 6, and method 7 in parallel by generating a large number of focused beams using a microlens array. All of the bright spots can be moved in the same trajectory, enabling parallel assembly of optical components. By optically accessing the interaction region from one or more sides of the container structure, two independently controlled multi-lens arrays can be used to move and manipulate a first set of one shape of particles to closely approach a second set of the same or another shape of particles, while preserving the desired orientation and relative alignment of each particle in a set.

In the previous methods, when two or more building blocks are being manipulated optically and begin to closely approach, it may be necessary to dramatically alter the spatial pattern of light near the particles, decrease the light intensity, or extinguish the light intensity altogether (e.g. by closing a shutter). For example, although two individual particles may be stable in two focused single beam optical traps that are well-separated, the combination of two particles and two optical traps in close proximity, whether the particles are bonded together in an assembly or unbonded in close proximity, may lead to an instability. To overcome the possible instability that can cause ejection of the particles from the trap(s), it may be necessary to block at least one of the beams once the particles are in close proximity. As another example, when two bright laser spots are being rapidly moved to manipulate two particles, as the particles begin to closely approach, it may be convenient to block one bright spot and cause the other bright spot to trace out the form of the completed assembly consisting of both particles.

For particles that exhibit a response to magnetic fields, applying at least one of magnetic fields or magnetic field gradients can be used to cause particles to migrate and even reorient in a desirable manner. This can be combined with optical methods and microfluidic methods of positioning and orienting particles to achieve a desired relative position and orientation of one building block relative to another one.

It is possible to position one microscope objective lens near one optical windows of the container structure and a second microscope objective lens on the opposite side of the container structure near the opposite optical window of the container structure so that at least a portion of the regions of manipulation and field of view of the two objective lenses overlap. This provides a means of trapping and manipulating building block particles to create an assembly using two different sources of focused light from opposing sides of the container structure.

Bonding Closely Proximate Building Blocks

Once brought into contact, the particles must be able to bind in either a sticky or slippery, or slidable manner so that thermal energy cannot drive them completely apart again. In some cases, it will be desirable to have moving parts, so slippery bonds that retain a lubricating layer of liquid between the particles, such as those induced by depletion attractions, will be preferred. In other embodiments, rigidly attaching the particles with a shear-rigid bond is preferred, and this can be achieved with a different type of interaction between the particles' surfaces.

In general, once the particles have been brought into close proximity, an attractive interaction must be present between the two particles. This attractive interaction can be formed in many ways.

Different types of interactions that can be used to effectively bond particles together include:

van der Waals attractions; Depletion attractions induced by nanoscale depletion agents such as (micelles, nanodroplets, nanoemulsions, nanoparticles, polymers, dendrimers, microgel particles, vesicles, biomolecules, surfactants); Surface roughness-controlled depletion attractions; Charge attractions; Entanglement of polymers between two surfaces; Microscopic or nanoscopic loops and barbs (e.g. velcro); Bridging of a material between the particles (usually a solid phase); Hydrophobic interactions; Hydrophilic interactions; Polar interactions; Bridging of biomolecules between surfaces; streptavidin-biotin; single-stranded DNA (ss-DNA) oligos, sections, or entire polymers having complimentary base pair sequences on one end and bonded to desired mating surfaces of two building blocks on the other end (e.g. through a derivatization process). (For example, ss-DNA having one terminal sequence is bonded to one or more surfaces of one particle, and ss-DNA having the complementary terminal sequence (in either forward or reverse order) is bonded to one or more surfaces of a second particle. When the complementary strands encounter one another, they line up to form a double helical strand and form a bond that has an energy that is significantly stronger than thermal energy.);

single-stranded DNA (ss-DNA) oligos, sections, or entire polymers having different base pair sequences on one end and bonded to desired mating surfaces of two building-block particles on the other end (e.g. through a derivatization process). In this case, a third linking ss-DNA strand is supplied in the fluid phase that contains within its structure both complementary base sequences of the exposed ends of both ss-DNA that have been attached to the particles. One terminal sequence is bonded to one or more surfaces of one particle, and ss-DNA having a different terminal sequence (in either forward or reverse order) is bonded to one or more surfaces of a second particle. When the linking ss-DNA is introduced into the solution, a portion of it attaches to each of the ss-DNA molecules bound to the particles' surfaces, forming a strong bond between the two particles.

double-stranded DNA (ds-DNA) can be used in the same manner as above with ss-DNA, except that a portion of the end of the molecule is only ss-DNA.

hand-and-glove interactions between biomolecules known to form dimers

membrane proteins

snare proteins

antibody-antigen

(this list of biomolecules is not exhaustive)

Bridging by synthetic molecules adsorbed on surfaces of particles

Encapsulation by membranes or coatings around particles

Encapsidation of particles

Growth of a condensed phase material over the surfaces of both particles

Growth of a condensed phase material between the surfaces of the particles

Chemical bonding of reactive groups on particles' surfaces (e.g. acidic and basic groups)

Photo-initiated bonding of reactive groups on the particles' surfaces

Creating attractions by altering the ionic strength or pH of the fluid phase

Altering the temperature to induce attractions

Altering the temperature to sinter particles

Swelling the particles so they touch

Casimir attractive forces

Using catalysts or initiators to induce polymerization of monomers in the fluid

Hydrogen bonding forces

Magnetic forces

Friction forces

Phase changes of materials in the fluid phase around the particles

Wetting of a liquid droplet onto surfaces of a solid particle

Surface tension of a wetting fluid between particles

Adding a chemical to the fluid around the particles that eliminates any repulsion and bonds the two particles' surfaces together

Closing links of particles that can form a chain

Inserting a part of one particle into another and then swelling or shrinking one or both particles

Interlocking particles by insertion of a part of one particle into another and then changing the shape of one or both of the particles

Screwing (twisting) one particle into another mating particle (e.g. screw cap)

Adding nanoparticles or macromolecules to the solution that bind with both surfaces of building-block particles to bond the building-block particles together

Supplying an external source of energy to bind particles together:

Electric voltage and current: electrodeposition

Chemical epitaxial growth

Electrochemical deposition

Microscopic laser welding: using a laser beam that causes the surfaces to react (e.g. an intense focused beam in the region between the two particles could melt the particle material and cause it to flow together before resolidifying). An appropriate beam can be a short (millisecond or less) high-energy pulse from a high-power laser through a lens.

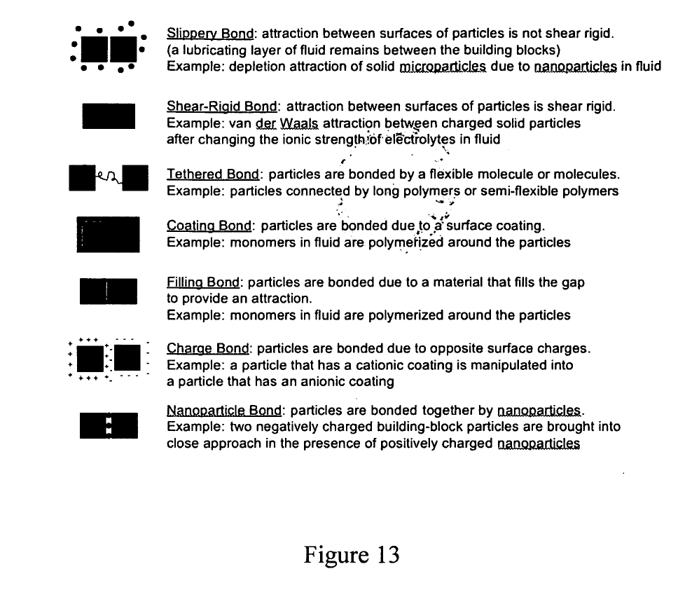

FIGS. 9-12 show examples of building blocks that are bonded together by several different methods after optical manipulation into close proximity. FIG. 13 illustrates some of the bonding methods listed in this section.

FIG. 9 is an example of creating an assembly of two lithographic particles using optical manipulation shown in a time series of optical micrographs (time increases from left to right in the top row, then continues to increase from left to right in the bottom row). A cross and a pentagon are brought in close proximity using a single beam gradient optical trap (helium neon laser, power P≈10 mW) and bonded together using a depletion attraction. The cross is trapped, reoriented, and moved with a single bright focused spot (small x) to the pentagon (which is partially constrained by a surface of the container structure) where it binds to the pentagon through a depletion attraction created by a nanoemulsion (droplet radius approximately 50 nm, droplet volume fraction in the fluid phase is approximately 15%). After the last frame shown, the laser beam is blocked and the particles remain together in the form of an assembly thereafter due to bonding by the depletion attraction. The end-to-end lengths of the crosses' arms are 4.5 microns.

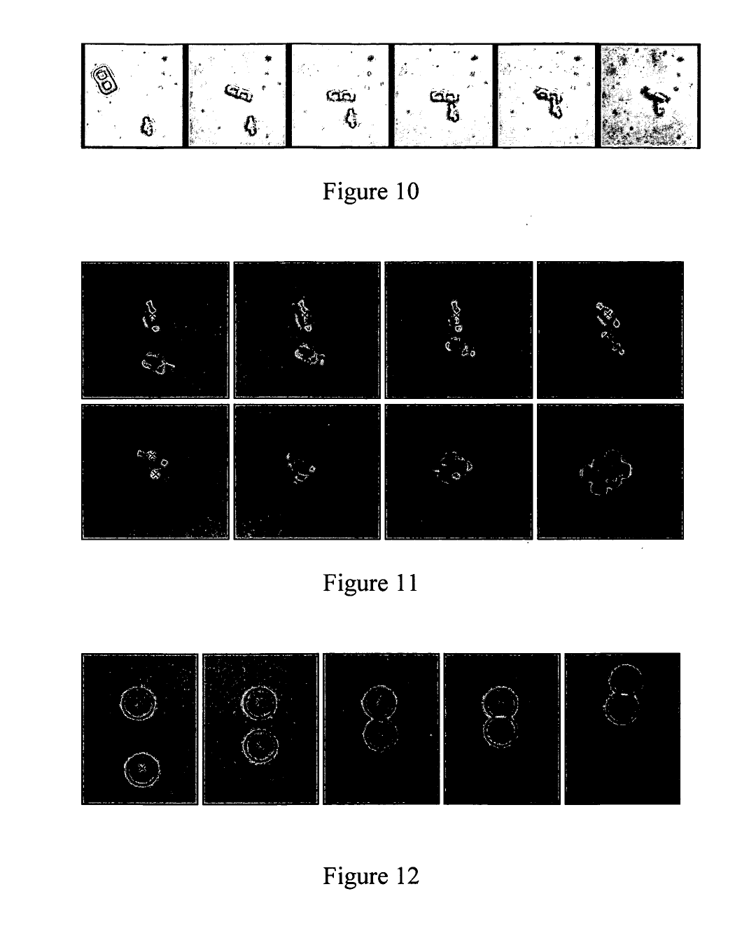

FIG. 10 shows a time sequence of optical micrographs of two lithographic microscale particles, a letter B and a square cross, that are moved into close proximity by the dual beam optical trap apparatus shown in FIG. 6 (with helium-neon lasers creating Gaussian TEM₀₀ beams, each at a power of about 10 mW). Time increases from left to right. The bright focused laser spot holding the B is stationary, and the bright focused laser spot holding the cross is moved with an electronically controlled piezoelectric mirror. The arm of the cross is inserted into the hole of the B. The length of the letter B is approximately 7 microns. The thickness of both particles is about 1 micron.

FIG. 11 is an example using two focused beam optical manipulation (see FIG. 6) to create an assembly of two building blocks consisting of identical square cross platelets that have been lithographically fabricated according to an embodiment of the current invention. The fluid material in which the crosses reside is an aqueous dispersion of nanoemulsion droplets having radius α≈50 nm and droplet volume fraction φ≈0.15. Both laser beams are helium-neon Gaussian TEM₀₀ at a power P≈10 mW focused through a Nikon CFI 100×1.4 numerical aperture microscope objective lens. The objective is also used to monitor and record the positions and orientations of the particles using a video camera connected to a computer. Green light illuminates the container structure through the glass plate on the opposite side of the container structure as the side occupied by the objective lens. The bright spots near the focal regions of the laser beams are indicated by X's. The progression of the assembly process is revealed as time increases from left to right in the top row of images and subsequently increases from left to right in the bottom row of images. The crosses are brought together in close approach as the top laser beam is moved toward the one in the center. As the cross particles approach closely, they bond together due to the depletion attraction to create an assembly. Subsequently, when both laser beams are blocked (note the lack of X's in the last few frames), the assembly does not separate under thermal fluctuations (e.g. see the last frame in the lower right).

FIG. 12 is an example of optical manipulation using two focused laser beams to assemble two sulfate-stabilized polystyrene microspheres (diameter of 3 microns) dispersed in water without any depletion agent according to an embodiment of the current invention. The apparatus schematic is shown in FIG. 6 and the equipment is described in more detail in FIG. 11. In this sequence of optical transmission micrographs, time increases from left to right. X's indicate the approximate location of the bright spot due to the strongly focused laser light. The spheres are brought into close approach when one bright spot is moved toward another using deflection caused by a piezoelectrically actuated mirror. Upon close approach (fourth frame from the left), a saline solution of sodium chloride at 1 M concentration is gently injected into the interaction region from a side channel without forcing the particles out of the trap, yielding an overall saline concentration of about 500 mM after the injection. The saline solution effectively defeats the repulsion of the negatively charged sulfate groups on the surfaces of the particles, and their solid surfaces bond as they enter the primary van der Waals minimum in their interaction potential. When the laser beams are blocked using a shutter (last frame at the far right), the spheres remain permanently bound together in a dumbbell assembly by a shear-rigid bond.

FIG. 13 shows examples of methods for creating attractive interactions (also called ‘bonds’) between building blocks that can be stronger than thermal energy so an assembly of building blocks will remain together after being brought into close approach according to some embodimentps of the current invention. A first building block (square on left) can be held together with a second building block (square on right) in a variety of ways. This list is not intended to be comprehensive, just illustrative. More examples of methods that can be used to hold particles together after manipulating them are described above.

Moving Assembled Building Blocks out of the Interaction Region

Once the assembly has been bonded so that the building blocks do not come apart when the optical and other external forces are removed, it is often desirable to transport the assembly out of the interaction region for use or storage in another location than the place it was fabricated. This can be accomplished using methods similar to those that have already been described for bringing the building blocks into the interaction region. A microfluidic container structure can have outlet channels, and fluid flows or other means can be used to move the assembly to a new region of the container structure. This new region may be a second interaction region at which an additional building block could be added to the assembly made in a first interaction region. This new region could also be a storage compartment that can hold the assemblies. Such storage compartments may be equipped with doors, gates, valves, or other means of allowing assemblies to enter or leave and for keeping the assemblies in the storage compartment over long periods of time.

Sub-Assemblies

Although the simplest form of an assembly is comprised of at least two building blocks, some assemblies may be comprised of many building blocks. For the case in which many building blocks must be assembled together, it can be useful and convenient to form a sub-assembly consisting of two or more building blocks as an intermediate step in putting together the complete assembly. Once formed, it is possible to treat a sub-assembly as a new kind of building block and apply the methods given in the previous sections for manipulating and further adding to the sub-assembly.

For instance, if a complete assembly consists of a total of four fundamental building blocks, a first sub-assembly of two building blocks can be created in a first interaction region, and a second sub-assembly of two building blocks can be created in a second interaction region. These two sub-assemblies can be transported to a third interaction region and further manipulated and bonded to form the complete assembly.

Examples of Assemblies

Many different kinds of assemblies can be fabricated using the methods described previously from a wide range of materials. Assemblies can have building blocks that are static or dynamic. The building blocks within an assembly can have completely fixed positions and orientations with respect to all of other building blocks in the assembly, or they can have some (usually limited) degrees of freedom to translate or rotate relative to all of the other building blocks. Assemblies that have at least one building block that can still translate or rotate relative to other building blocks after assembly is complete are considered to have internal moving parts. Dimensions of fabricated assemblies are typically less than a few millimeters, although they could be larger in principle. Examples of complex shapes that can be used as building blocks or colloidal structural components include: letters, numbers, symbols, gears, wedges, screws, nuts, bolts, lids, cups, spikes, cones, truncated cones, screw tops, levers, angles, multi-point stars, multi-arm stars, plates, valves, filters, tubes, collars, plungers, paddles, rudders, axles, spindles, wheels, washers, toriods, elastic springs, elastic coils, bearings, nozzles, mating hinge pieces, and spacers. Small-scale functional objects that can be fabricated through the assembly processes described herein can include, but are not limited to: cups, caps, containers, bottles, joints (universal, hinge, ball-and-socket, dovetail), stamp, stamping template, lithographic template (optical, mechanical, electrochemical), platforms, ramps, levers, pulleys, screws, nuts, bolts, nails, spikes, wrenches, washers, keys, knives, knobs, sorters, sieves, saws, transmissions, clutches, brakes, wipers, ball-bearings, cross roller bearings, separators, stand-offs, bevel gears, gears, wheels, elastic springs, rotators, carts, fuselage, wings, ailerons, rudders, motors (e.g. combustion, electrical, or magnetic—brushless, DC, servo), worm drives, pistons, pumps, valves, hinges, lights, wheels, tracks, locks, doors, windows, chains, sliders, frames, latches, transistors, electronic devices, computer electronic devices, processors, microprocessors, thermometers, heaters, coolers, batteries, diodes, capacitors, inductors, resistors, electrical lines, memory storage devices, photovoltaic devices, thermovoltaic devices, electrically-insulated electrical connectors, terminal connectors, electrically insulated cables, plugs, sockets, speakers, switches (mechanical, electrical, optical), sound generators, electricity generators, turbines, bubble generators, droplet generators, particle generators, wave generators, beacons, propellers, translational stages, rotational stages, tip-tilt stages, mechanical jacks, optical component holders, scissors, stairs, elevators, escalators, rollers, conveyors, vehicles, crawlers, robots, piezoelectric actuators, light emitting diodes, microscopic lasers, scanning probe microscopy devices, combs, antennae, transmitters of electromagnetic radiation, receivers of electromagnetic radiation, concentrators of electromagnetic radiation, membranes, porous media, mechanical filters, electrical filters, optical filters, barbs, photonic band-gap materials, biological cell storage devices, biological cell encapsulation systems, mechanical structures to limit or guide biological cell growth and biological cell motility, and customized tissues comprised of biological cellular building blocks. Energy can be supplied to propel moving parts of assemblies from molecules, nanoparticles, or droplets that are dispersed in the fluid around the assemblies.

As an example of a powered assembly, a building block can have a biological motor and propulsion system, such as a flagellum extracted from the membrane of a bacterium, attached to its surface and this can consume energy from molecules (e.g. ATP and GTP) in solution to move a portion of an assembly or the entire assembly.

One potential application of some embodiments of the current invention is to build microbottles and trap nanoscale polymers inside them; such bottles might have potential microscopic drug delivery applications. The exposed surfaces of flat caps (square platelets) are coated with negatively charged sulfate groups. By contrast, the exposed rims of the cups would be treated with positively charged amine groups. Each of these dispersions (i.e. caps and cups) can exist in separate input reservoirs without aggregation because of the similar sign charge for that particular building block. When a cap and a cup are manipulated into close approach in the presence of a desired biologically functional molecule in the fluid, the caps will close on the cups, forming an assembly that prevents the escape of the molecules from the bottles. Other biologically relevant bonding agents, such as streptavidin and biotin, could also be used to close the bottles. Opening the bottles could then be triggered by the introduction of specific enzymes that can cleave these linkages. With microbottles based on this principle, it might be possible to deliver cell-killing drug molecules in a targeted manner to areas in the body that have an abundance of certain enzymes (e.g. caused by cancer) when these enzymes unwittingly open the microbottles.

1. A system for producing multi-component colloidal structures, comprising:
a supply system;
an assembly system that is in fluid connection with said supply system to receive a supply of colloidal structural components from said supply system; and
an output system in fluid connection with said assembly system,
wherein said assembly system comprises an assembly chamber adapted to contain colloidal structural components during assembly of a multi-component colloidal structure, and
wherein said assembly system is structured and arranged to control positions and orientations of first and second colloidal structural components in said assembly chamber to bring said first and second colloidal structural components together in predetermined relative positions and orientations for assembly into at least a portion of said multi-component colloidal structure.

2. A system for producing multi-component colloidal structures according to claim 1, further comprising an imaging system constructed and arranged to observe said first and second colloidal structural components in at least one of said supply system, said assembly system and said output system while said system for producing multi-component colloidal structures is in operation.

3. A system for producing multi-component colloidal structures according to claim 2, wherein said imaging system includes automated image acquisition and image processing functions.

4. A system for producing multi-component colloidal structures according to claim 2, wherein said imaging system is a video microscopy imaging system that provides signals for real-time computer-controlled feedback to facilitate bringing said first and second colloidal structural components into said assembly system, a control of relative positions and orientations of said first and second colloidal structural components in said assembly system, and an exiting of said at least a portion of said multi-component colloidal structure from said assembly system.

5. A system for producing multi-component colloidal structures according to claim 1, wherein said supply system is constructed to supply said first and second colloidal structural components to said assembly chamber in at least one of a selectable position or orientation with respect to said assembly system.

6. A system for producing multi-component colloidal structures according to claim 5, wherein said supply system comprises a microfluidic chip that has microchannels to deliver predetermined colloidal structural components to said assembly chamber.

7. A system for producing multi-component colloidal structures according to claim 1, wherein said supply system comprises a component production system that is constructed to be suitable to produce said supply of colloidal structural components such that they have substantially predetermined sizes and shapes.

8. A system for producing multi-component colloidal structures according to claim 1, wherein said first and second colloidal structural components each have a maximum dimension that is greater than about 30 nm and less than about 100 m.

9. A system for producing multi-component colloidal structures according to claim 1, wherein at least one of said first and second colloidal structural components is a lithographically produced colloidal structural component.

10. A system for producing multi-component colloidal structures according to claim 1, wherein at least one of said first and second colloidal structural components has a complex shape that can be described by a Reeb graph that contains at least one of a loop, a branch, a node, or an arc.

11. A system for producing multi-component colloidal structures according to claim 1, wherein said first colloidal structural component has at least a portion of a surface that is adapted to mate with at least a portion of a surface of said second colloidal structural component when said first and second colloidal structural components are brought together in predetermined relative positions and orientations.

12. A system for producing multi-component colloidal structures according to claim 1, wherein said assembly system further comprises an optical system that is structured and arranged to at least assist in said controlling positions and orientations of said first and second colloidal structural components in said assembly chamber to bring said first and second colloidal structural components together for said assembly into at least said portion of said multi-component colloidal structure.

13. A system for producing multi-component colloidal structures according to claim 12, wherein said optical system is structured and arranged to produce optical intensity patterns in said assembly chamber such that said intensity patterns are selected based on shapes of at least a portion of each of said first and second colloidal structural components to provide optical traps to trap and manipulate said first and second colloidal structural components.

14. A system for producing multi-component colloidal structures according to claim 13, wherein said supply system is constructed to supply said first and second colloidal structural components to said assembly chamber in at least one of a selectable position or orientation with respect to said optical system.

15. A system for producing multi-component colloidal structures according to claim 13, wherein information about a size and shape of at least one of said first and second colloidal structural components from said imaging system is used at least in part to select and generate said intensity patterns by said optical system.

16. A system for producing multi-component colloidal structures according to claim 13, wherein said optical system comprises an optical source and an optical adjustment component arranged to intercept said optical source in a path between said optical source and at least one of said first and second colloidal structural components when said first and second colloidal structural components are in said assembly chamber.

17. A system for producing multi-component colloidal structures according to claim 16, wherein said optical adjustment component comprises a spatial light modification element constructed to form a predetermined intensity pattern holographically in said assembly chamber to at least assist in said controlling positions and orientations of said first and second colloidal structural components.

18. A system for producing multi-component colloidal structures according to claim 16, wherein said optical adjustment component comprises a light steering element that is actuated to form a predetermined intensity pattern in said assembly chamber to at least assist in said controlling positions and orientations of said first and second colloidal structural components.

19. A system for producing multi-component colloidal structures according to claim 16, wherein said optical system comprises a first laser as said optical source to provide illumination at a first wavelength and a second laser to provide illumination at a second wavelength.

20. A system for producing multi-component colloidal structures according to claim 1, further comprising a bonding material injection system in fluid connection with said assembly system, wherein said bonding material injection system is adapted to provide a material that causes said first and second colloidal structural components when held in selected relative positions and orientations to be bonded together.

21. A system for producing multi-component colloidal structures according to claim 1, wherein at least one of viscous forces, fluid forces, electromagnetic forces, optical forces, magnetic forces, electrophoretic forces, dielectrophoretic forces, gravitational forces, buoyant forces, viscous torques, fluid torques, electromagnetic torques, optical torques, magnetic torques, electrophoretic torques, dielectrophoretic torques, gravitational torques, buoyant torques are used to overcome forces and torques due to thermal fluctuations, thereby providing a means to position and orient at least one of said first and second colloidal structural components.

22. A method of producing multi-component colloidal structures, comprising:
controlling a position and an orientation of a first colloidal structural component;
controlling a position and an orientation of a second colloidal structural component; and
bringing said first and second colloidal structural components together into predetermined relative positions and orientations to be assembled into at least a portion of a multi-component colloidal structure.

23. A method of producing multi-component colloidal structures according to claim 22, wherein said controlling positions and orientations of said first and second colloidal structural components comprise optical trapping of said first and second colloidal structural components.

24. A method of producing multi-component colloidal structures according to claim 23, wherein said optical trapping said first and second colloidal structural components comprises forming optical intensity patterns that are selected based on shapes of at least a portion of each of said first and second colloidal structural components.

25. A method of producing multi-component colloidal structures according to claim 22, further comprising forming a bond between said first and second colloidal structural components to form a multi-component colloidal structure.

26. A method of producing multi-component colloidal structures according to claim 25, further comprising injecting a bonding material into a vicinity of said first and second colloidal structural components to cause them to bond together.

27. A method of producing multi-component colloidal structures according to claim 25, further comprising illuminating said first and second colloidal structural components with a localized laser pulse to bond said first and second colloidal structural components together in a desired relative position and orientation in a manner of flash welding.

28. A method of producing multi-component colloidal structures according to claim 25, wherein said bond is a substantially rigid bond.

29. A method of producing multi-component colloidal structures according to claim 25, wherein said bond is a slippery bond that permits at least some motion between said first and said second colloidal structural components.

30. A method of producing multi-component colloidal structures according to claim 25, further comprising:
controlling a position and an orientation of a third colloidal structural component; and
bringing said third colloidal structural component and said multi-component colloidal structure together into predetermined relative positions and orientations to be assembled into at least a portion of another multi-component colloidal structure.

31. A multi-component colloidal structure produced according to the method of claim 22.
